SEQUENCE LISTING (5 sequences)

SEQ ID NO: 1 - DNA, 18 bp, Artificial sequence
  Synthetic sequence Primer AmGSTLF1
atggccgcagctgcagca

SEQ ID NO: 2 - DNA, 22 bp, Artificial sequence
  Synthetic sequence Primer AmGSTLF2
ggtgccttccctggagcacgac

SEQ ID NO: 3 - DNA, 18 bp, Artificial sequence
  Synthetic sequence Primer AmGSTLR1
gtcgtgctccagggaagg

SEQ ID NO: 4 - DNA, 25 bp, Artificial sequence
  Synthetic sequence Primer AmGSTLR2
ccaagctaaattggccaaggaagaa

SEQ ID NO: 5 - protein, 10 aa, Alopecurus myosuroides
VFGPAMSTNV

(19) United States
(12) Patent Application Publication
(10) Pub. No.: US 2010/0184601 A1
(43) Pub. Date: Jul. 22, 2010

(54) Method and Means Relating to Multiple Herbicide Resistance in Plants

(75) Inventor: Ian Cummins, Durham (GB)

Correspondence Address: Tod T. Tumey, P.O. BOX 22188, HOUSTON, TX, 77227-2188, US

(73) Assignee: University of Durham, Durham, Durham (GB)

(21) Appl. No.: 12/678,153

(22) PCT Filed: Sep. 15, 2008
(86) PCT No.: PCT/GB2008/050826

§ 371 Date: Mar. 30, 2010

(30) Foreign Application Priority Data
Sep. 14, 2007 (GB) 0717982.3

(51) Int. Cl. A01N 43/832; A01N 43/84; A01N 43/66; C12Q 1/18; C12Q 1/48; C07D 271/12; C07D 413/10; C07D 285/14; A01P 13/00

(52) U.S. Cl. 504/227; 504/265; 504/224; 504/261; 435/32; 435/15; 548/126; 544/138

(57) ABSTRACT

Methods for overcoming multiple herbicide resistance (MHR) in plants using inhibitors of GST suppression of Formula (I), novel chemical inhibitors of Formula (Ia), compositions comprising compounds of Formula (I), and uses and methods relating thereto.

### FIELD OF INVENTION

The present invention relates to methods for overcoming multiple herbicide resistance (MHR) in plants, uses and methods relating thereto and compounds therefore. In particular, the invention relates to methods for overcoming MHR in weed species belonging to the Gramineae wherein benzodiazole compounds are applied thereto.

### BACKGROUND OF INVENTION

The acquisition of herbicide resistance by weed populations is a global problem with serious implications to sustainable arable agriculture (1-3). The best characterised resistance mechanisms arise from mutations in proteins that are targeted by herbicides rendering the proteins less sensitive to inhibition. Such target site-based resistance (TSR) confers tolerance to all herbicides sharing that mode of action. Mutations leading to TSR have been well described for acetyl CoA carboxylases (ACCases, 4) involved in fatty acid metabolism, acetolactate synthase (ALS; branched chain amino acid biosynthesis, 5) and the plastoquinone binding protein of photosystem II (PSII; photosynthesis, 6). While TSR in weeds is widespread, the mutations underpinning it tend to confer a fitness penalty, such that on removing the herbicide selection pressure, the resistance trait is steadily lost in the field (2). More practically, TSR can be countered by rotating herbicide usage such that compounds with alternative modes of action are employed to restore chemical control (1).

A second and more problematic mechanism is based on weeds acquiring multiple herbicide resistance (MHR). This is distinct from herbicide cross-resistance, which can arise from the pyramiding of multiple TSR traits (3, 6). Instead, MHR weeds deploy a central defense system, which counteracts herbicide-imposed toxicity irrespective of the original site of action. MHR appears to be linked to an enhanced ability to detoxify herbicides and as such it has also been termed metabolism-based resistance (3, 7, 8). MHR is most problematic in the grass weeds associated with cereal crops; notably black-grass (Alopecurus myosuroides), wild oat (Avena fatua), and annual rye-grass (Lolium rigidum) (1). Because of similarities in physiology and biochemistry, selective control of grasses is always a major challenge in cereals, being reliant upon differential rates of herbicide detoxification (9). By enhancing weed metabolism, MHR neutralizes the selectivity mechanism of graminicidal herbicides, allowing wild grasses to compete more effectively with cereals and leading to major losses in crop yield and quality (1-3). In view of the threat posed to the sustainability of arable agriculture, overcoming MHR in grass weeds has become the subject of international research efforts co-ordinated through agencies such as the herbicide resistance action committee (HRAC; www.plantprotection.org/HRAC/).

MHR in black-grass was first reported in 1982 at the Peldon site in Essex, England (10). Independent outbreaks have since been reported around the world and include a characterized MHR black-grass population in Cordoba, Spain (7). The enhanced ability of MHR black-grass to metabolize herbicides is due to elevated levels of herbicide detoxifying enzymes such as cytochrome P450 mixed function oxidases (CYPs, 11), glutathione transferases (GSTs, 12, 13) and O-glucosyltransferases (OGTs, 14).

U.S. Pat. No. 6,495,370B1 describes haloenol lactone compounds that are alleged to be useful for preventing herbicide resistance in plants. Such compounds are stated as allegedly being enzyme inhibitors of glutathione transferases (GSTs) in plants, although no data are provided to support this allegation. All the data relate to GSTs from murine sources and inter alia, the inhibition thereof. It is clear from statements found in this patent that the potential for combating resistance through inhibition of plant GSTs would be restricted to herbicides which are directly detoxified by these enzymes. The inhibitors described in U.S. Pat. No. 6,495,370B1 are not acting to interfere with a causative agent of MHR and are unlike the inhibitors contemplated herein which act on a class of GSTs that co-ordinate the MHR phenotype in plants. Thus, the inhibitors used in the methods of the instant invention neither appear to be of the same chemical type as, nor do they appear to act in the same manner as, those of U.S. Pat. No. 6,495,370B1.

WO87/04596 describes herbicidal compositions which comprise a constituent that “partially inhibits activity of plant enzymes which take part in the pathway of detoxification of active oxygen species of plants”. The invention described in this patent application is another example wherein components of the herbicidal composition claimed do not appear to act on an enzyme that is a causative agent of MHR, but on enzyme(s) that are responsible for down-stream toxic events resulting from target site-based resistance.

It has now been found that in the case of the GSTs, a single enzyme that was named AmGST2a (from Alopecurus myosuroides; accession AJ010451), now re-named AmGSTF1-1, was highly expressed in all MHR black-grass populations tested, but not in herbicide-susceptible wild type (WT) or TSR plants (15). This enzyme, AmGSTF1-1, is active as a dimer formed from AmGSTF1 subunits, and is a member of the so-called plant-specific phi (F) class of GSTs (16). AmGSTF1-1 showed little activity in detoxifying herbicides, but was very active as an antioxidant glutathione peroxidase (GPOX, 15). It has further been found that by regulating or controlling the activity of certain glutathione transferases in plant cells, such as AmGSTF1-1, plants that would normally display MHR toward herbicides can become susceptible to such herbicides. In such cases, the MHR response is substantially weakened, reduced or abolished relative to the MHR response found in plants of the same species that display MHR and wherein GST activity has not been otherwise regulated or controlled.

Advantages of the invention will become apparent from the following description.

### SUMMARY OF INVENTION

According to the present invention there is provided a method for selectively controlling multiple herbicide resistance (MHR) in weed plants in the field, the method comprising applying to plants in the field at least one chemical inhibitor of Formula (I)

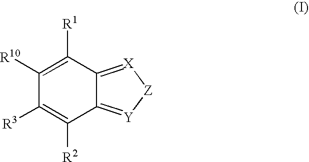

wherein
R¹ is selected from H, (C₁-C₁₅) alkyl, (C₁-C₁₅)haloalkyl, NO₂, SO₂NR⁴R⁵, SO₂R⁶, SO₂V, SO₂NH(CH₂)₁₋₆CONH—NHCOV, SO₂NH(CH₂)₁₋₆CONH—N═CHV, CHO, COOR⁷, CONR⁴R⁵, Br, Cl, F, CH═CHCOO(CH₂)ₙCH₃, CN, SO₂(—NTN—)(CH₂)nV, SO₂N═SR⁸R⁹V, SO₂OV, COV, and (C₃-C₉) heteroaryl ring containing at least one of O, N, and S, C₆ aryl ring, C₁₀ aryl ring, wherein the said heteroaryl ring, said C₆ aryl ring and said C₁₀ aryl ring are optionally substituted with COOR⁷;
R² is selected from H, F, Cl, Br, (C₁-C₆)alkyl, (C₁-C₆)haloalkyl, NR⁴R⁵, OR⁴, SR⁴, S(CH₂)ₙOH, S(CH₂)ₙCOOR⁷, CH═CHCOOR⁷, CN, O(CH₂)ₙOR⁷, O(C₁-C₆)alkylCOOH, NHCO(C₁-C₆)alkyl, NHCO(C₆aryl), NHCO(C₁₀aryl), NHCO(heteroaryl ring), and a S(C₃-C₉) heteroaryl ring containing at least one of N, O, and S, optionally substituted with H, (C₁-C₆)alkyl, F, Br, C₁, NO₂, NHR⁴ or NR⁴R⁵;
R³ is selected from CF₃, NO₂ and H;
R⁴ and R⁵ are independently selected from H, (C₁-C₁₅) alkyl, (C₁-C₁₅) haloalkyl, (CH₂)N₃, a C₆-aryl ring, a C₁₀aryl ring, a (C₃-C₉) heteroaryl ring containing at least one of O, S and N wherein the said C₆aryl ring, said C₁₀aryl ring, and said (C₃-C₉) heteroaryl ring are optionally substituted with at least one of OH, Cl, Br, F, CF₃, COO(C₁-C₆)alkyl, (C₁-C₆) alkyl; or R⁴ and R⁵ together form a 4-8 membered heterocyclic ring structure containing carbon atoms and optionally at least one ring member selected from O, S and N;
R⁶ is selected from H, (C₁-C₁₅) alkyl, OH, Cl, Br, and F;
R⁷ is selected from H, (C₁-C₆) alkyl, C₆-aryl ring, a C₁₀aryl ring, a (C₃-C₉) heteroaryl ring containing at least one of O, S and N wherein the said C₆aryl ring, said C₁₀aryl ring, and said (C₃-C₉) heteroaryl ring are optionally substituted with at least one of OH, Cl, Br, F, CF₃, COO(C₁-C₆)alkyl, and (C₁-C₆) alkyl;
R⁸ is ═O;
R⁹ is (C₁-C₆) alkyl;
R¹⁰ is selected from H, Cl, Br, F, (C₁-C₁₅) alkyl, (C₁-C₁₅) haloalkyl, SR⁴, and NR⁴R⁵;

(—NTN—) is a piperazine ring structure;
V is selected from a (C₃-C₉) heteroaryl ring containing at least one of O, S and N, a C₆aryl ring, a C₁₀aryl ring wherein the said (C₃-C₉) heteroaryl ring, said C₆ aryl ring and said C₁₀aryl ring are optionally substituted with at least one of (C₁-C₆) alkyl, CF₃, O, Br, Cl, and F;
X is selected from N and N⁺—O⁻;
Y is selected from N and N⁺—O⁻;
Z is selected from O, Se and S; and
n is a whole integer selected from 1 to 8.
Preferably, the method for selectively controlling multiple herbicide resistance (MHR) in weed plants in the field, comprises applying to plants in the field at least one chemical inhibitor of Formula (I)

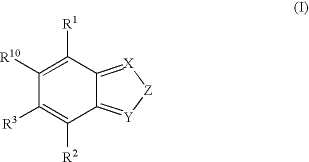

wherein:
R¹ is selected from H, (C₁-C₁₀) alkyl, (C₁-C₁₀) haloalkyl, NO₂, SO₂NR⁴R⁵, SO₂R⁶, SO₂V, SO₂NH(CH₂)₁₋₆CONH—NHCOV, SO₂NH(CH₂)₁₋₆CONH—N═CHV, CHO, COOR⁷, CONR⁴R⁵, Br, Cl, F, CH═CHCOO(CH₂)CH₃, CN, SO₂(—NTN—)(CH₂)nV, SO₂N═SR⁸R⁹V, SO₂OV, COV, and (C₄-C₇) heteroaryl ring containing at least one of O, N, and S, C₆ aryl ring, wherein the said heteroaryl ring, and said C₆ aryl ring are optionally substituted with COOR⁷;
R² is selected from H, F, Cl, Br, (C₁-C₆)alkyl, (C₁-C₆)haloalkyl, NR⁴R⁵, OR⁴, SR⁴, S(CH₂)ₙOH, S(CH₂)ₙCOOR⁷, CH═CHCOOR⁷, CN, O(CH₂)—OR⁷, O(C₁-C₆)alkylCOOH, NHCO(C₁-C₆)alkyl, NHCO(C₆aryl), NHCO(C₆aryl), NHCO(heteroaryl ring), and a S(C₃-C₉) heteroaryl ring containing at least one of N, O, and S, optionally substituted with H, (C₁-C₆)alkyl, F, Br, C₁, NO₂, NHR⁴ or NR⁴R⁵;
R³ is selected from CF₃, NO₂ and H;
R⁴ and R⁵ are independently selected from H, (C₁-C₁₀) alkyl, (C₁-C₁₀) haloalkyl, (CH₂)ₙN₃, a C₆-aryl ring, a (C₄-C₈) heteroaryl ring containing at least one of O, S and N wherein the said C₆aryl ring, and said (C₄-C₈) heteroaryl ring are optionally substituted with at least one of OH, Cl, Br, F, CF₃, COO(C₁-C₆)alkyl, (C₁-C₆) alkyl; or R⁴ and R⁵ together form a 4 or 5 membered heterocyclic ring structure containing carbon atoms and optionally at least one ring member selected from O, S and N;
R⁶ is selected from H, (C₁-C₆) alkyl, OH, Cl, Br, and F;
R⁷ is selected from H, (C₁-C₆) alkyl, C₆-aryl ring, a (C₄-C₈) heteroaryl ring containing at least one of O, S and N wherein the said C₆aryl ring, and said (C₄-C₈) heteroaryl ring are optionally substituted with at least one of OH, Cl, Br, F, CF₃, COO(C₁-C₆)alkyl, and (C₁-C₆) alkyl;
R⁸ is ═O;
R⁹ is (C₁-C₆) alkyl;
R¹⁰ is selected from H, Cl, Br, F, (C₁-C₁₀) alkyl, (C₁-C₁₀) haloalkyl, SR⁴, and NH₂;
(—NTN—) is a piperazine ring structure;
V is selected from a (C₃-C₉) heteroaryl ring containing at least one of O, S and N, a C₆aryl ring, wherein the said (C₃-C₉) heteroaryl ring and said C₆ aryl ring are optionally substituted with at least one of (C₁-C₄) alkyl, CF₃, O, Br, Cl, and F;
X is selected from N and N⁺—O⁻;
Y is selected from N and N⁺—O⁻;
Z is selected from O, Se and S; and
n is a whole integer selected from 1 to 8.

More preferably, the method for selectively controlling multiple herbicide resistance (MHR) in weed plants in the field comprises applying to plants in the field at least one chemical inhibitor of Formula (I)

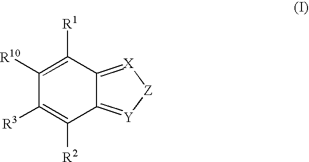

wherein:
R¹ is selected from H, (C₁-C₆) alkyl, (C₁-C₆) haloalkyl, NO₂, SO₂NR⁴R⁵, SO₂R⁶, SO₂V, SO₂NH(CH₂)₁₋₄CONH—NHCOV, SO₂NH(CH₂)₁₋₄CONH—N═CHV, CHO, COOR⁷, CONR⁴R⁵, Br, Cl, F, CH═CHCOO(CH₂)CH₃, CN, SO₂(—NTN—)(CH₂)nV, SO₂N═SR⁸R⁹V, SO₂OV, COV, and (C₄-C₇) heteroaryl ring containing at least one of O, N, and S, C₆ aryl ring, wherein the said heteroaryl ring, and said C₆ aryl ring are optionally substituted with COOR⁷;
R² is selected from H, F, Cl, Br, (C₁-C₆)alkyl, (C₁-C₆)haloalkyl, NR⁴R⁵, OR⁴, SR⁴, S(CH₂)ₙOH, S(CH₂)ₙCOOR⁷, CH═CHCOOR⁷, CN, O(CH₂)ₙOR⁷, O(C₁-C₆)alkylCOOH, NHCO(C₁-C₆)alkyl, NHCO(C₆aryl), NHCO(heteroaryl ring), and a S(C₃-C₉) heteroaryl ring containing at least one of N, O, and S, optionally substituted with H, (C₁-C₆)alkyl, F, Br, C₁, NO₂, NHR⁴ or NR⁴R⁵;
R³ is selected from CF₃, NO₂ and H;
R⁴ and R⁵ are independently selected from H, (C₁-C₆) alkyl, (C₁-C₆) haloalkyl, (CH₂)ₙN₃, a C₆-aryl ring, a (C₄-C₈) heteroaryl ring containing at least one of O, S and N wherein the said C₆aryl ring, and said (C₄-C₈) heteroaryl ring are optionally substituted with at least one of OH, Cl, Br, F, CF₃, COO(C₁-C₆)alkyl, (C₁-C₆) alkyl; or R⁴ and R⁵ together form a 4 or 5 membered heterocyclic ring structure containing carbon atoms and optionally at least one ring member selected from O, S and N;
R⁶ is selected from H, (C₁-C₆) alkyl, OH, Cl, Br, and F;
R⁷ is selected from H, (C₁-C₄) alkyl, C₆-aryl ring, a (C₄-C₈) heteroaryl ring containing at least one of O, S and N wherein the said C₆aryl ring, and said (C₄-C₈) heteroaryl ring are optionally substituted with at least one of OH, Cl, Br, F, CF₃, COO(C₁-C₄)alkyl, and (C₁-C₄) alkyl;
R⁸ is ═O;
R⁹ is (C₁-C₆) alkyl;
R¹⁰ is selected from H, Cl, Br, F, (C₁-C₁₀) alkyl, (C₁-C₁₀) haloalkyl, SR⁴, and NH₂;
(—NTN—) is a piperazine ring structure;
V is selected from a (C₃-C₇) heteroaryl ring containing at least one of O, S and N, a C₆aryl ring, wherein the said (C₃-C₇) heteroaryl ring and said C₆ aryl ring are optionally substituted with at least one of (C₁-C₄) alkyl, CF₃, O, Br, Cl, and F;
X is selected from N and N⁺—O⁻;
Y is selected from N and N⁺—O⁻;
Z is selected from O, Se and S; and
n is a whole integer selected from 1 to 6.

Still more preferably, the method for selectively controlling multiple herbicide resistance (MHR) in weed plants in the field comprises applying to plants in the field at least one chemical inhibitor of Formula (I)

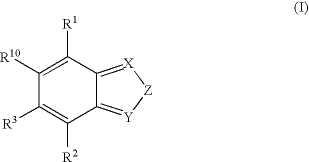

wherein:
R¹ is selected from H, (C₁-C₃) alkyl, (C₁-C₃) haloalkyl, NO₂, SO₂NR⁴R⁵, SO₂R⁶, SO₂V, SO₂NH(CH₂)₁₋₄CONH—NHCOV, SO₂NH(CH₂)₁₋₄CONH—N═CHV, CHO, COOR⁷, CONR⁴R⁵, Br, Cl, F, CH═CHCOO(CH₂)₄CH₃, CN, SO₂(—NTN—)(CH₂)nV, SO₂N═SR⁸R⁹V, SO₂OV, COV, and (C₄-C₇) heteroaryl ring containing at least one of O, N, and S, C₆ aryl ring, wherein the said heteroaryl ring, and said C₆ aryl ring are optionally substituted with COOR⁷;
R² is selected from H, F, Cl, Br, (C₁-C₃)alkyl, (C₁-C₃)haloalkyl, NR⁴R⁵, OR⁴, SR⁴, S(CH₂)₆OH, S(CH₂)₂COOR⁷, CH═CHCOOR⁷, CN, O(CH₂)₂OR⁷, O(C₁-C₆)alkylCOOH, NHCO(C₁-C₆)alkyl, NHCO(C₆aryl), NHCO(heteroaryl ring), and a S(C₃-C₉) heteroaryl ring containing at least one of N, O, and S, optionally substituted with H, (C₁-C₆)alkyl, F, Br, C₁, NO₂, NHR⁴ or NR⁴R⁵;
R³ is selected from CF₃, NO₂ and H;
R⁴ and R⁵ are independently selected from H, (C₁-C₄)alkyl, (C₁-C₄)haloalkyl, (CH₂)₄N₃, a C₆-aryl ring, a (C₄-C₈)heteroaryl ring containing at least one of O, S and N wherein the said C₆aryl ring, and said (C₄-C₈) heteroaryl ring are optionally substituted with at least one of OH, Cl, Br, F, CF₃, COO(C₁-C₆)alkyl, (C₁-C₆) alkyl; or R⁴ and R⁵ together form a 4 or 5 membered heterocyclic ring structure containing carbon atoms and optionally at least one ring member selected from O, S and N;
R⁶ is selected from H, (C₁-C₆) alkyl, OH, Cl, Br, and F;
R⁷ is selected from H, (C₁-C₄)alkyl, C₆-aryl ring, a (C₄-C₈) heteroaryl ring containing at least one of O, S and N wherein the said C₆aryl ring, and said (C₄-C₈) heteroaryl ring are optionally substituted with at least one of OH, Cl, Br, F, CF₃, COO(C₁-C₃)alkyl, and (C₁-C₄) alkyl;
R⁸ is ═O;
R⁹ is (C₁-C₆) alkyl;
R¹⁰ is selected from H, Cl, Br, F, (C₁-C₆) alkyl, (C₁-C₆) haloalkyl, SR⁴, and NH₂;
(—NTN—) is a piperazine ring structure;
V is selected from a (C₃-C₇) heteroaryl ring containing at least one of O, S and N, a C₆aryl ring, wherein the said (C₃-C₇) heteroaryl ring and said C₆aryl ring are optionally substituted with at least one of CF₃, O, Br, Cl, and F;
X is selected from N and N⁺—O⁻;
Y is selected from N and N⁺—O⁻;
Z is selected from O, Se and S; and
n is a whole integer selected from 1 to 6.

Yet more preferably, the method for selectively controlling multiple herbicide resistance (MHR) in weed plants in the field comprises applying to plants in the field at least one chemical inhibitor of Formula (I)

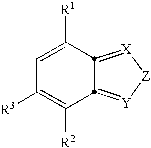

wherein
R¹ is selected from H, (C₁-C₃) alkyl, (C₁-C₃) haloalkyl, NO₂, SO₂NR⁴R⁵, SO₂R⁶, SO₂V, SO₂NH(CH₂)₁₋₄CONH—NHCOV, SO₂NH(CH₂)₁₋₄CONH—N═CHV, CHO, COOR⁷, CONR⁴R⁵, Br, Cl, F, CH═CHCOO(CH₂)₄CH₃, CN, SO₂(—NTN—)(CH₂)nV, SO₂N═SR⁸R⁹V, SO₂OV, COV, and (C₄-C₇) heteroaryl ring containing at least one of O, N, and S, C₆ aryl ring, wherein the said heteroaryl ring, and said C₆ aryl ring are optionally substituted with COOR⁷;
R² is selected from H, F, Cl, Br, (C₁-C₃)alkyl, (C₁-C₃)haloalkyl, NR⁴R⁵, OR⁴, SR⁴, S(CH₂)₆OH, S(CH₂)₂COOR⁷, CH═CHCOOR⁷, CN, O(CH₂)₂OR⁷, O(C₁-C₆)alkylCOOH, NHCO(C₁-C₆)alkyl, NHCO(C₆aryl), NHCO(heteroaryl ring), and a S(C₃-C₉) heteroaryl ring containing at least one of N, O, and S, optionally substituted with H, (C₁-C₆)alkyl, F, Br, C₁, NO₂, NHR⁴ or NR⁴R⁵;
R³ is selected from CF₃, NO₂ and H;
R⁴ and R⁵ are independently selected from H, (C₁-C₄) alkyl, (C₁-C₄) haloalkyl, (CH₂)₄N₃, a C₆-aryl ring, a (C₄-C₈) heteroaryl ring containing at least one of O, S and N wherein the said C₆aryl ring, and said (C₄-C₈) heteroaryl ring are optionally substituted with at least one of OH, Cl, Br, F, CF₃, COO(C₁-C₆)alkyl, (C₁-C₆) alkyl; or R⁴ and R⁵ together form a 4 or 5 membered heterocyclic ring structure containing carbon atoms and optionally at least one ring member selected from O, S and N;
R⁶ is selected from H, (C₁-C₆) alkyl, OH, Cl, Br, and F;
R⁷ is selected from H, (C₁-C₄) alkyl, C₆-aryl ring, a (C₄-C₈) heteroaryl ring containing at least one of O, S and N wherein the said C₆aryl ring, and said (C₄-C₈) heteroaryl ring are optionally substituted with at least one of OH, Cl, Br, F, CF₃, COO(C₁-C₃)alkyl, and (C₁-C₄) alkyl;
R⁸ is ═O;
R⁹ is (C₁-C₆) alkyl;
(—NTN—) is a piperazine ring structure;
V is selected from a (C₃-C₇) heteroaryl ring containing at least one of O, S and N, a C₆aryl ring, wherein the said (C₃-C₇) heteroaryl ring and said C₆aryl ring are optionally substituted with at least one of CF₃, O, Br, Cl, and F;
X is selected from N and N⁺—O⁻;
Y is selected from N and N⁺—O⁻;
Z is selected from O, Se and S; and
n is a whole integer selected from 1 to 6.

Compounds of Formula (I) have been observed to affect the activity of at least one glutathione transferase (GST) that is capable of conferring MHR to a plant or of at least one catalytically active subunit thereof.

It should be understood that the terms “alkyl” and “haloalkyl” refer to either substituents or parts of substituents as defined for Formula (I) and Formula (Ia), depending on context. “Alkyl” refers to a straight chain or branched chain or cyclic alkyl where appropriate, such as cyclopropyl, cyclopentyl and cyclohexyl and the like. Thus, “alkyl” may include up to fifteen carbon atoms in the chain, that is, from (C₁-C₁ₛ) alkyl, preferably from (C₁-C₁₀)alkyl and more preferably (C₁-C₆)alkyl, as provided for in the definition of Formula (I). Suitable examples may be selected from methyl, ethyl, prop-1-yl, prop-2-yl, the cyclopropyl group, butyl, isobutyl, sec-butyl, tert-butyl, pentyl, isopentyl, neopentyl, straight chain hexyl, 2-methylpentyl, 3-methylpentyl, 2,3-dimethyl butyl, 2,2-dimethylbutyl, cyclopentyl, and cyclohexyl, where appropriate. “Haloalkyl” conforms to the definition provided for “alkyl” as provided above but wherein the haloalkyl chain or haloalkyl substitutent comprises at least one halo substituent located thereon that is selected from Cl, F, and Br.

Most preferably, the method for selectively controlling multiple herbicide resistance (MHR) in weed plants in the field, comprises applying to plants in the field at least one chemical inhibitor of Formula (I) selected from compounds i) to lxvii), inclusive:

### Compound i) 4-Chloro-7-nitrobenzo[c][1,2,5]oxadiazole (4-chloro-7-nitro-2,1,3-benzoxadiazole)

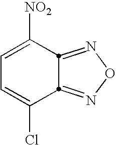

### Compound ii) 4-Methoxy-7-nitrobenzo[c][1,2,5]oxadiazole (4-methoxy-7-nitro-2,1,3-benzoxadiazole)

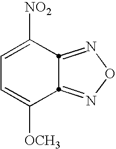

### Compound iii) 4-Nitro-7-(pyrrolidin-1-yl)benzo[c][1,2,5]oxadiazole (4-nitro-7-(1′-pyrrolidinyl)-2,1,3-benzoxadiazole)

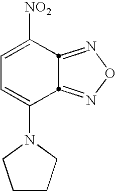

### Compound iv) 7-Chloro-N-methylbenzo[c][1,2,5]oxadiazole-4-sulfonamide (4-(methylsulfonamido)-7-chloro-2,1,3-benzoxadiazole)

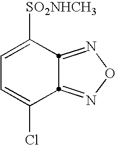

### Compound v) N-(4-azidobutyl)-7-chlorobenzo[c][1,2,5]oxadiazole-4-sulfonamide (4-(4′-azidobutylsulfonamido)-7-chloro-2,1,3-benzoxadiazole)

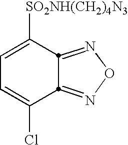

### Compound vi) 4-Fluoro-7-nitrobenzo[c][1,2,5]oxadiazole (4-fluoro-7-nitro-2,1,3-benzoxadiazole)

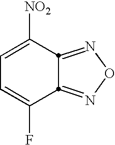

### Compound vii) 4,6-Dinitrobenzo[c][1,2,5]oxadiazole 1-oxide (4,6-dinitro-2,1,3-benzoxadiazole-1-oxide)

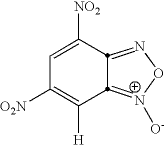

### Compound viii) 4-bromo-7-nitrobenzo[c][1,2,5oxadiazole

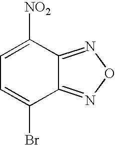

### Compound ix) 4-(methylthio)-7-nitrobenzo[c][1,2,5oxadiazole

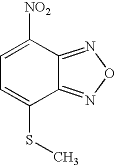

### Compound x) 4-Nitro-7-(4-trifluoromethyl)phenylthio)benzo[c][1,2,5oxadiazole

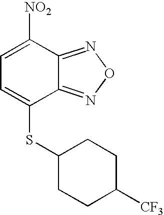

### Compound xi) 4-morpholino-7-nitrobenzo[c][1,2,5oxadiazole

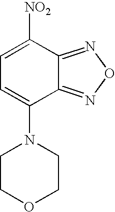

### Compound xii) 4-ethoxy-7-nitrobenzo[c][1,2,5oxadiazole

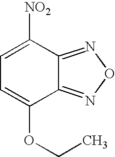

### Compound xiii) 7-Nitrobenzo[c][1,2,5oxadiazole

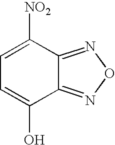

### Compound xiv) 3-(7-Nitrobenzo[c][1,2,5oxadiazol-4-ylthio)propanoic acid

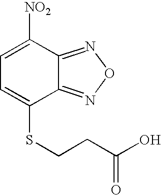

### Compound xv) 6-(7-Nitrobenzo[c][1,2,5oxadiazol-4-ylthio)hexan-1-ol

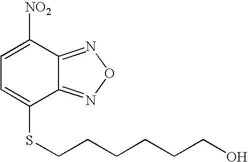

### Compound xvi) 7-bromo-N-propylbenzo[c][1,2,5thiadiazole-4-carboxamide

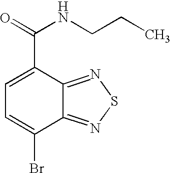

### Compound xvii) 7-bromo-N-methylbenzo[c][1,2,5thiadiazole-4-carboxamide

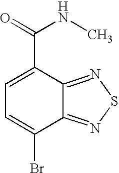

### Compound xviii) 7-bromo-N,N-dimethylbenzo[c][1,2,5 thiadiazole-4-carboxamide

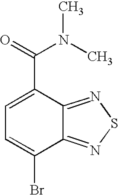

### Compound xix) Methyl 7-bromobenzo[c][1,2,5thiadiazole-4-carboxylate

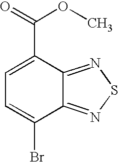

### Compound xx) Methyl 4-(7-bromobenzo[c][1,2,5thiadiazole-4-yl)benzoate

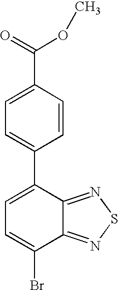

### Compound xxi) 4-Bromo-7-nitrobenzo[c][1,2,5thiadiazole

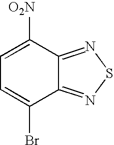

### Compound xxii) 4-Bromo-7-nitrobenzo[c][1,2,5selenadiazole

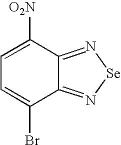

### Compound xxiii) 7-Bromobenzo[c][1,2,5thiadiazole-4-carboxylic acid

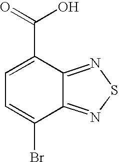

### Compound xxiv) (2E,′2E)-Dibutyl 3,3′-(benzo[c][1,2,5thiadiazole-4,7-diyl)diprop-2-enoate

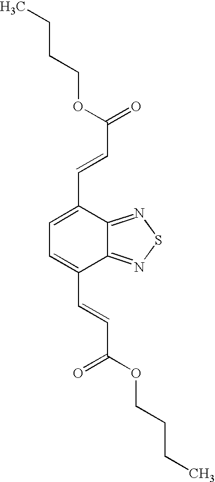

### Compound xxv) 4-Methylbenzo[c][1,2,5selenadiazole

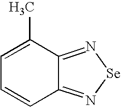

### Compound xxvi) 4-Bromo-7-methylbenzo[c][1,2,5selenadiazole

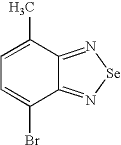

### Compound xxvii) Benzo[c][1,2,5selenadiazole-4,7-dicarbonitrile

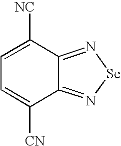

### Compound xxviii) 4,7-Dibromobenzo[c][1,2,5oxadiazole

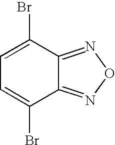

### Compound xxix) 2-(7-nitrobenzo[c][1,2,5oxadiazol-4-yloxy)ethanol

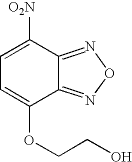

### Compound xxx) 4-Nitrobenzo[c][1,2,5oxadiazole

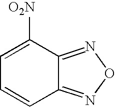

### Compound xxxi) Methyl 4-(7-bromobenzo[c][1,2,5selenadiazol-4-yl)benzoate

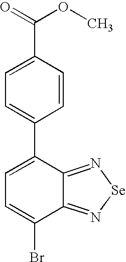

### Compound xxxii) 4-Nitro-7-phenoxybenzo[c][1,2,5oxadiazole

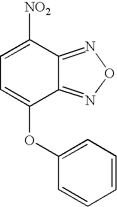

### Compound xxxiii) 7-Chloro-N,N-dimethylbenzo[c][1,2,5oxadiazole-4-sulfonamide

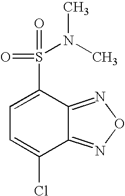

### Compound xxxiv) 4-Bromobenzo[c][1,2,5oxadiazole

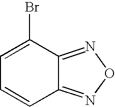

### Compound xxxv) 4-Nitro-7-(piperidin-1-yl)benzo[c][1,2,5oxadiazole

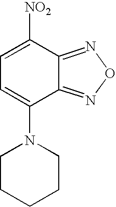

### Compound xxxvi) 5-Chloro-4-nitrobenzo[c][1,2,5thiadiazole

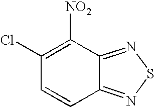

### Compound xxxvii) 7-Chloro-4-nitrobenzo[c][1,2,5oxadiazole 1-oxide

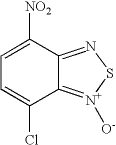

### Compound xxxviii) 4-Nitro-7-phenoxybenzo[c][1,2,5oxadiazole

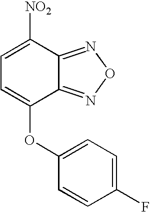

### Compound xxxix) N-Methyl-7-nitrobenzo[c][1,2,5oxadiazol-4-amine

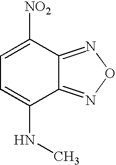

### Compound xl) N-(7-Nitrobenzo[c][1,2,5oxadiazol-4-yl)ethanamide

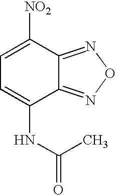

### Compound xli) 4-(Methyl(7-nitrobenzo[c][1,2,5oxadiazol-4-yl)amino)phenyl

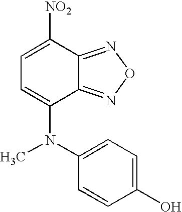

### Compound xlii) 7-Methyl-4-nitrobenzo[c][1,2,5oxadiazole 1-oxide

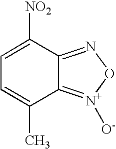

### Compound xliii) 5,7-Dinitrobenzo[c][1,2,5oxadiazole 1-oxide

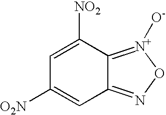

### Compound xliv) 4-(5,7-Dinitrobenzo[c][1,2,5oxadiazol-4-ylamino)phenol

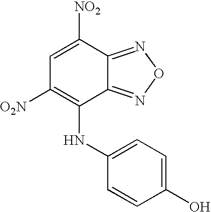

### Compound xlv) Methyl 4-(5,7-dinitrobenzo[c][1,2,5oxadiazol-4-ylamino)benzoate

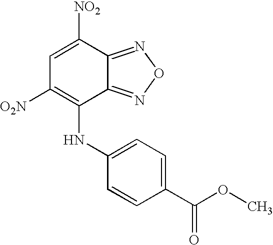

### Compound xlvi) 7-Bromo-5-methyl-4-nitrobenzo[c][1,2,5oxadiazole

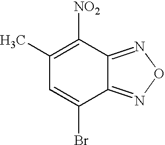

### Compound xlvii) N,N-dipropylbenzo[c][1,2,5thiadiazole-4-sulfonamide

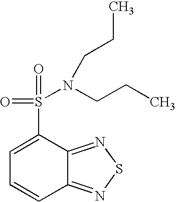

### Compound xlviii) 4-(Benzo[d]thiazol-2-ylthio)-7-nitrobenzo[c][1,2,5thiadiazole

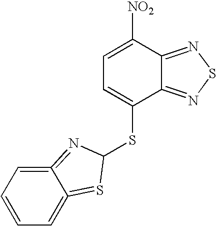

### Compound xlix) Bis(7-nitrobenzo[c][1,2,5thiadiazol-4-yl)sulfane

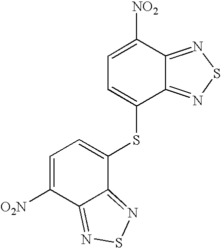

### Compound l) 5-(4-Chlorophenylthio)-4-nitrobenzo[c][1,2,5thiadiazole

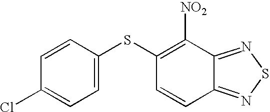

### Compound li) 5,7-Dinitrobenzo[c][1,2,5thiadiazol-4-amine

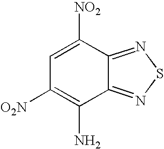

### Compound lii) 5-Chloro-4-nitrobenzo[c][1,2,5selenadiazole

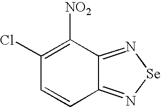

### Compound liii) 4-(7-Morpholinobenzo[c][1,2,5thiadiazol-4-ylsulfonyl)morpholine

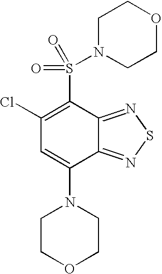

### Compound liv) 4-Nitrobenzo[c][1,2,5thiadiazole

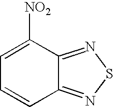

### Compound lv) (E)-N-(2-(2-(4-Chlorobenzylidene)hydrazinyl)-2-oxoethyl)benzo[c][1,2,5thiadiazole-4-sulfonamide

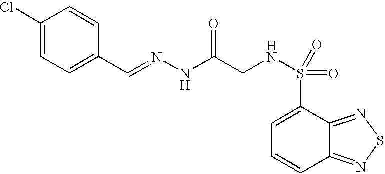

### Compound lvi) (E)-N-(2-(2-(2-Chlorobenzylidene)hydrazinyl)-2-oxoethyl)benzo[c][1,2,5thiadiazole-4-sulfonamide

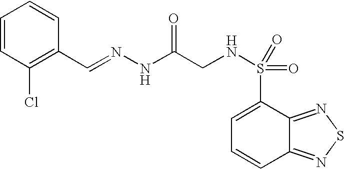

### Compound lxvii) N-(2-(2-(2-Chlorophenylcarbonyl)hydrazinyl)-2-oxoethyl)benzo[c][1,2,5thiadiazole-4-sulfonamide

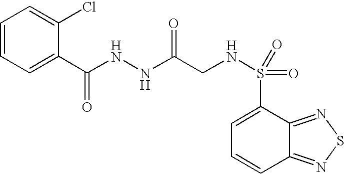

### Compound lviii) N-(2-(2-(4-Chlorophenylcarbonyl)hydrazinyl)-2-oxoethyl)benzo[c][1,2,5thiadiazole-4-sulfonamide

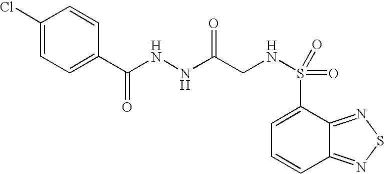

### Compound lix) N-(2-Oxo-2-(2-(3 trifluoromethyl)phenylcarbonyl)hydrazinyl)ethyl)benzo [c][1,2,5thiadiazole-4-sulfonamide

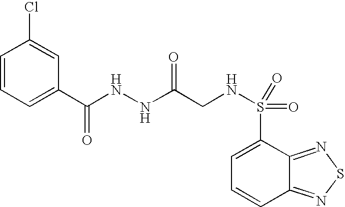

### Compound lx) 4-(4-(4-Chlorophenethyl)piperazin-1-ylsulfonyl)benzo [c][1,2,5thiadiazole-4-sulfonamide

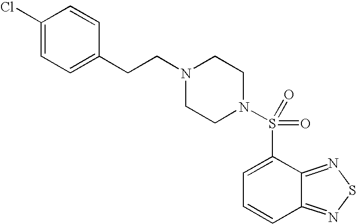

### Compound lxi) S-Methyl-5-phenyl-N-(benzo[c][1,2,5oxadiazolyl-4-sulfonyl)sulfoximine

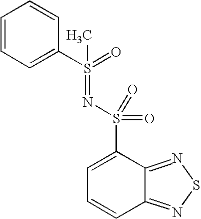

### Compound lxii) 3,5-Dichlorophenylbenzo[c][1,2,5thiadiazole-4-sulfonate

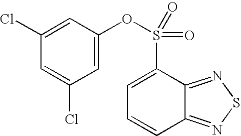

### Compound lxiii) 4-Chlorophenyl benzo[c][1,2,5thiadiazole-4-sulfonate

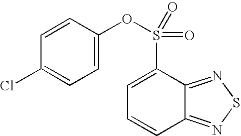

### Compound lxiv) 4-Nitrobenzo[c][1,2,5oxadiazol-5-amine

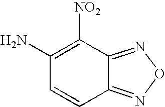

### Compound lxv) 4-(2-Chloro-4-(trifluoromethyl)phenoxy)-7-nitrobenzo[c]-[1,2,5oxadiazole

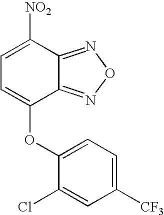

### Compound lxvi) 7-Nitro-N,N-dipropylbenzo[c][1,2,5oxadiazol-4-amine

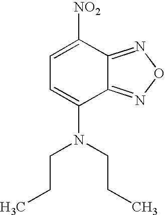

### Compound lxvii) Benzo[c][1,2,5oxadiazole

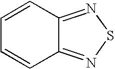

For the purposes of the present invention, the terms “weed plants” and “weed plant” refer to plants that compete with crop plants in the field. The terms are used interchangeably unless context demands otherwise. MHR weed plants are plants of species that are typically of the Gramineae and/or of the Poaceae that have acquired herbicide resistance from being exposed or contacted with more than one type of herbicide over time. Thus as a preferred embodiment there is provided a method for selectively controlling multiple herbicide resistance (MHR) in weed plants of the Gramineae and/or of the Poaceae in a field, the method comprising applying to said weed plants in the field at least one chemical inhibitor of Formula (I) that is effective in regulating the activity of at least one glutathione transferase or an active subunit thereof that is capable of conferring MHR to a plant. Preferably, the chemical inhibitor capable of regulating the activity of a relevant glutathione transferase or an active subunit thereof is selected from compounds i) to lxvii).

“MHR weed plants” are plants that display multiple herbicide resistance to at least two herbicides with differing modes of action that are applied to plants in the field, such as toward graminicides, for example, selective graminicidal herbicides such as the phenyl urea graminicides (inhibitors of photosystem II), such as chlortoluron, the aryloxyphenoxypropionate graminicides (acetyl CoA carboxylase inhibitors), such as fenoxaprop ethyl, the cyclohexanediones, such as pinoxaden, the sulfonyl ureas, such as iodosulfuron methyl, the triazines, such as atrazine and the like. Examples of weed plants wherein MHR has been observed include black-grass (Alopecurus myosuroides), wild oat (Avena fatua), and annual rye-grass (Lolium rigidum) all of which may be considered to be weed plants appropriate for treating with inhibitors in methods of the present invention. Other MHR weed plants able to be controlled by methods of the present invention include species of grasses from the families Echinochloa, Setaria, Sorghum, Phalaris and Bromus.

The inhibitor can be any chemical that is capable of regulating, typically decreasing, the level of activity of at least a GST that is capable of conferring MHR to a plant or plant cell, or at least one active subunit thereof that is capable of conferring MHR to a plant or plant cell. Preferably the chemical is one that is selected from those encompassed by Formula (I) and Formula (Ia) (novel compounds) as herein defined. For the avoidance of doubt the term “GST” as referred to herein means a GST or appropriate active subunit(s) thereof that is(are) capable of conferring MHR to a plant cell or a plant, unless context demands otherwise. Thus, dimers of GSTs, monomers of GSTs, individual GST subunits, and/or combinations of GST subunits that are capable of conferring MHR to a plant cell or a plant are encompassed within the ambit of “GST” as used in methods and uses of the invention, unless context demands otherwise. GST activity that is referred to herein encompasses enzyme activity, that is to say a catalytic activity and/or the ability to regulate the activity of other proteins through binding interactions. GST activity is thought to be selectively controlled, typically decreased, by contact of the chemical inhibitor with a GST that is able to confer MHR to a plant, such that MHR is significantly reduced or abolished. Conventional herbicides that are substantially ineffective on plants that display MHR may be applied to treated plants in conjunction with chemical inhibitor or after application of the inhibitor, as described below. A suitable class of chemicals that has been found to be active in suppressing MHR in black-grass are the benzodiazoles, and most notably the benzo[c][1,2,5]diazoles such as the benzo[c][1,2,5]oxadiazoles, benzo[c][1,2,5]thiadiazoles, benzo[c][1,2,5]selenadiazoles (also referred to in the art as 2,1,3-benzoxadiazoles, 2,1,3-benzthiadiazoles, and 2,1,3-benzselenadiazoles) for example, the chemical inhibitors of Formula (I), and especially the benzo[c][1,2,5]diazoles numbered from i) to lxvii) herein. Accordingly there is provided use of at least one chemical inhibitor according to Formula (I) in the suppression of MHR in weed plants. In a preferred aspect, there is provided use of at least one chemical inhibitor according to Formula (I) in the suppression of MHR in weed plants that is selected from the benzo[c][1,2,5]diazoles numbered from i) to lxvii) herein. More preferably, there is provided use of at least one chemical inhibitor according to Formula (I) in the suppression of MHR in weed plants that is selected from the chemical compounds numbered i) to lxvii) herein.

The GST or at least one catalytically active subunit thereof that the inhibitor acts upon must be one that acts as at least a causative agent of MHR in a plant, such as a weed plant or a transgenic plant comprising an introduced functional GST enzyme or functional part thereof that is capable of conferring MHR on a transformed plant cell or a whole plant. Thus, MHR can be observed in plants in which two or more herbicides with differing modes of action are applied to plants in the field without serious deleterious effect to the viability of the plants. Causative agents of MHR in plants include the GSTs, such as the phi class of GSTs, for example, AmGSTF1-1 or at least one functional subunit thereof. By “functional” is meant that the GST, such as AmGSTF1-1, or a subunit thereof is capable of conferring MHR on a plant. Naturally, the skilled addressee will appreciate that such functional homologues of AmGSTF1-1 are included within the ambit of the invention as are orthologues of AmGSTF1 such as those from Triticum species, Oryza species, Hordeum species, Avena species, and others. Examples of orthologues that appear to encode AmGSTF1-like proteins include proteins in wheat (TaGST19E50 & TaGSTF6 accession numbers AY064481, AJ440795), barley (HvGST6, AF430069) and rice (OsGSTF1, OsGSTF8, NP_{—}001065199, and NM_{—}193836. Based on similarities in protein cross-reactivity with an antiserum that recognizes AmGSTF1, further orthologues are also present in monocot grass weeds from the families Echinochloa, Setaria, Sorghum, Phalaris and Bromus.

The method of selective control, that is to say, the application of the GST chemical inhibitor of choice as described herein, typically reduces GST activity thus weakening or abolishing MHR in weeds which have acquired MHR traits through repeated herbicide use in the field. Following treatment with a GST inhibitor, such as one of Formula (I) and/or (II), the formerly resistant weeds are rendered susceptible to herbicides that under normal, conventional circumstances would not have had a deleterious effect on the viability of untreated plants. Preferably, the method for selectively controlling MHR is a method for down-regulating MHR-inducing activity in target plants.

In a further aspect of the invention there is provided a method for selectively controlling the viability of plants displaying MHR in a field that comprises:

i) contacting the said plants with a chemical inhibitor of a GST that confers GST-mediated MHR to the plants; and

ii) contacting the said plants with at least one herbicide.

Preferably, there is provided a method for selectively controlling the viability of plants displaying MHR in a field that comprises:

i) contacting the said plants with at least one chemical inhibitor of a GST of Formula (I) that confers GST-mediated MHR to the plants; and

ii) contacting the said plants with at least one herbicide.

More preferably, there is provided a method for selectively controlling the viability of plants displaying MHR in a field that comprises:

i) contacting the said plants with at least one chemical inhibitor of a GST of Formula (I) that is selected from compounds i) to vii) that confers GST-mediated MHR to the plants; and

ii) contacting the said plants with at least one herbicide.

More preferably the compounds used in this aspect of the invention are selected from compounds of Formula (I) numbered from i) to lxvii) herein.

The application of herbicide to plants in the field, such as weed plants, can be at a time prior to, during, or after the application of the inhibitor, depending on the formulation of the inhibitor and herbicide of choice. Typically, the inhibitor is applied prior to the application of herbicide within a time interval wherein the inhibition of the GST by the applied inhibitor is effective to render the plant susceptible to applied herbicide thereafter. The time interval may be up to 48 hours or more in duration depending on the dosage strength of the inhibitor, route of uptake and transport of the inhibitor by the plant, the plant species, timing of application, formulation of the inhibitor and environmental conditions. Typically, the time interval is measured in the order of up to several hours, for example, up to 8 hours, from application of the chemical inhibitor. Alternatively, the herbicide can be administered simultaneously with the application of inhibitor depending on the herbicide, manner of application of inhibitor and/or herbicide and other parameters suggested herein. For example, the inhibitor could be co-applied with herbicide if formulated so as to allow the sequential release of the inhibitor followed by the herbicide. Typically, the herbicide is applied to plants in the field after a period of time in which the reducing or abolishing effect of the inhibitor on the causative enzyme activity leading to MHR has taken effect but without leaving such a long period that the inhibitor's effect is weakened by the removal of the compound or the turnover and replacement of the inhibited GST.

In a further embodiment of the invention there is provided a method for identifying a GST inhibitor for use in the control of MHR weed plants in a field comprising i) isolating a plant cell from a plant that displays MHR; ii) applying an organic chemical to the plant cell; iii) applying a first herbicide to the said plant cell; and iv) analysing the said plant cell for viability. Preferably, the GST inhibitor is one of Formula (I). Such a screening method may be applicable to whole plants or populations of cells obtained from a whole plant.

Accordingly, there is provided a method for screening a plant that displays MHR that comprises i) applying an organic chemical to the plant; ii) applying a first herbicide to the said plant; and iii) analysing the plant for viability. “Plant” in the context of this embodiment of the invention encompasses a whole plant or a population of cells obtained from a plant that displays MHR. The test plant cell or plant can be either from a transformed plant or a plant that has acquired MHR through exposure to herbicides in the field. “A suitable GST inhibitor” is one that can be used in methods of the invention for controlling weed plant infestation and will be limited to those that control the activity of the GST but which do not have a substantially deleterious effect on the viability of the crop plant, transformed plant or plant cell(s) of interest. Such GST inhibitors are preferably selected from one or more of those benzo[c][1,2,5]diazoles of Formula (I), such as from the group of numbered compounds i) to lxvii) as provided herein. Compounds which provide protection against a first herbicide may then be re-tested with a second herbicide having a mode of action distinct from that of the first herbicide to confirm the ability of the compound to inhibit MHR. Thus, there is also provided a method for identifying a GST inhibitor for use in the control of MHR weed plants in a field that comprises i) isolating a plant cell from a plant that displays MHR; ii) applying an organic chemical to the plant cell; iii) applying at least two herbicides having a different modes of action to the said plant cell; and iv) analysing the said plant cell for viability. The skilled addressee will also appreciate that herbicides to be tested on a plant cell may be added together or at discrete time intervals one after the other. Thus in a preferment, there is provided a method for identifying a GST inhibitor for use in the control of MHR weed plants in a field that comprises i) isolating a plant cell from a plant that displays MHR; ii) applying an organic chemical to the plant cell; iii) applying a first herbicide having a first mode of action to the said plant cell; iv) analysing the said plant cell for viability; v) adding a second herbicide having a mode of action different to that of the first herbicide to a viable plant cell obtained from step iv); and vi) analyzing the plant cell for viability. Naturally, the skilled addressee will appreciate that a similar sequence of steps may be performed on a whole plant or population of plant cells obtained from a plant that displays MHR. The skilled addressee will also appreciate that herbicides to be tested on a plant may be added together or at discrete time intervals one after the other.

Accordingly, there is provided a method for screening a plant that displays MHR that comprises i) applying an organic chemical to the plant; ii) applying at least two herbicides having different modes of action to the plant; iii) analyzing the plant for viability. In a refinement of this embodiment there is provided a method for screening a plant that displays MHR that comprises i) applying an organic chemical to the plant; ii) applying a first herbicide to the said plant; iii) analysing the plant for viability; iv) adding a second herbicide having a mode of action different to that of the first herbicide to a viable plant obtained from step iii); and iv) analyzing the said plant for viability.

As another embodiment of the invention there is provided a method for identifying a GST chemical inhibitor by screening an isolated GST derived from a plant that displays MHR that comprises i) measuring GST activity; ii) contacting a chemical compound with the isolated GST; iii) measuring GST activity after contact with the said chemical compound; and iv) comparing the GST activity measured under step i) with that of step iii). Naturally, the man skilled in the art will appreciate that any enzyme inhibitor activity found under the in vitro method outlined above would need to be tested on live plant cells or live plants for suitability for use in methods of the invention.

In a further embodiment of the invention there is provided a compound of Formula (Ia):

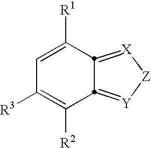

wherein
R¹ is selected from NO₂, CONR⁴R⁵, CN, SO₂NR⁴R⁵, COOR⁴, CONR⁴R⁵, and a C₆aryl ring optionally substituted with COOR⁴;
R² is selected from H, F, Cl, Br, CN, NHR⁴, NR⁴R⁵, OR⁴, and SR⁴;
R³ is selected from NO₂ and H;
R⁴ and R⁵ are independently selected from H, (C₁-C₄)alkyl, (CH₂)ₙN₃ or a C₆aryl ring optionally substituted with CF₃, or a C₆-aryl ring, a (C₄-C₇) heteroaryl ring containing at least one of O, S and N wherein the said C₆aryl ring, and said (C₄-C₇) heteroaryl ring are optionally substituted with at least one of OH, Cl, Br, F, CF₃, COO(C₁-C₆)alkyl, (C₁-C₆)alkyl; or R⁴ and R⁵ together form a 5 membered heterocyclic ring structure containing carbon atoms and optionally at least one ring member selected from O, S and N;
X is selected from N and N⁺—O⁻;
Y is selected from N and N⁺—O⁻;
Z is selected from O, Se and S.
Preferred novel compounds of Formula (II) include compounds iv), v), xvi)-xx), xxvii), and xxxi):

### Compound iv) 7-Chloro-N-methylbenzo[c][1,2,5]oxadiazole-4-sulfonamide (4-(methylsulfonamido)-7-chloro-2,1,3-benzoxadiazole)

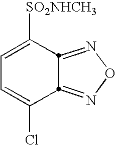

### Compound v) N-(4-azidobutyl)-7-chlorobenzo[c][1,2,5]oxadiazole-4-sulfonamide (4-(4′-azidobutylsulfonamido)-7-chloro-2,1,3-benzoxadiazole)

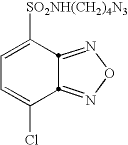

### Compound xvi) 7-bromo-N-propylbenzo[c][1,2,5thiadiazole-4-carboxamide

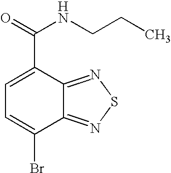

### Compound xvii) 7-bromo-N-methylbenzo[c][1,2,5thiadiazole-4-carboxamide

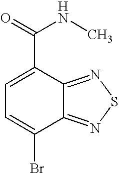

### Compound xviii) 7-bromo-N,N-dimethylbenzo[c][1,2,5 thiadiazole-4-carboxamide

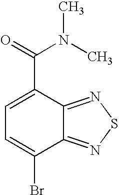

### Compound xix) Methyl 7-bromobenzo[c][1,2,5thiadiazole-4-carboxylate

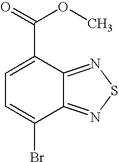

### Compound xx) Methyl 4-(7-bromobenzo[c][1,2,5thiadiazole-4-yl)benzoate

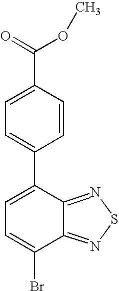

### Compound xxvii) Benzo[c][1,2,5selenadiazole-4,7-dicarbonitrile

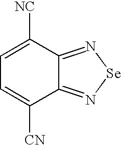

### Compound xxxi) Methyl 4-(7-bromobenzo[c][1,2,5selenadiazol-4-yl)benzoate

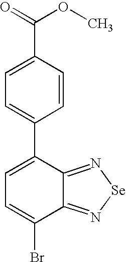

The compounds of Formula (I), that is to say inhibitors, such as inhibitors selected from the group of benzoxadiazoles e.g. selected from compounds i) to lxvii) herein, are useful in reducing or abolishing MHR in plants that display MHR, such as black-grass (Alopecurus myosuroides), wild oat (Avena fatua), and annual rye-grass (Lolium rigidum). The inhibitors may also be useful in reducing or abolishing MHR in transgenic plants that express whole GSTs or transgenic plants that express at least one catalytically active subunit thereof that is capable of conferring MHR on a plant. Suitable inhibitors of MHR in plants as demonstrated herein include compounds such as those listed herein.

Known inhibitors of GSTs that confer MHR to plants can be applied alone or in a mixture with other plant regulators, fertilizers, pesticides, herbicides, or fungicides. Suitable inhibitors can be applied along with an herbicide or prior to addition of herbicide. The inhibitor may be applied in a mixture with a carrier or, if necessary, other auxiliary agents to form any one of the standard types of preparations commonly used in agriculture, for example, a dust, granules, grains, a wettable powder, an emulsion, an aqueous solution etc.

Suitable solid carriers are clay, talc, kaolin, bentonite, terra abla, calcium carbonate, diatomaceous earth, silica, synthetic calcium silicate, kieselguhr, dolomite, powdered magnesia, Fuller's earth, gypsum and the like. Solid compositions comprising a suitable inhibitor may also be in the form of dispersible powders or grains, comprising in addition to the active ingredient, a surfactant to facilitate the dispersion of the powder or grains in liquid.

Liquid compositions include solutions, dispersions or emulsions containing at least one active suitable GST inhibitor together with one or more surface-active agents such as wetting agents, dispersing agents, emulsifying agents, or suspending agents.

Surface-active agents may be of the cationic, anionic, or non-ionic type. Suitable agents of the cationic type include, for example, quaternary ammonium compounds. Suitable agents of the anionic type include, for example, soaps such as Triton® X-100 and Tween® 20; salts of aliphatic mono-esters of sulphuric acid, for example sodium lauryl sulphate; and salts of sulphonated aromatic compounds, for example sodium dodecyl-benzenesulphonate, sodium, calcium, and ammonium lignosulphonate, butylnaphthalene sulphonate, and a mixture of the sodium salts of diisopropyl- and triisopropyl-naphthalene-sulphonic acid. Suitable agents of the non-ionic type include, for example, the condensation products of ethylene oxide with fatty alcohols such as oleyl alcohol and cetyl alcohol, or with alkyl phenols such as octylphenol, nonylphenol, and octylcresol. Other non-ionic agents are the partial esters derived from long chain fatty acids and hexitol anhydrides, for example sorbitanmonolaurate; the condensation product of the said partial esters with ethylene oxide; and the lecithins.

Suitable suspending agents are, for example, hydrophilic colloids, for example polyvinylpyrrolidone and sodium carboxymethylcellulose, and the vegetable gums, for example, gum acacia and gum tragacanth. Preferred detergents are polyoxyethylenesorbitan (monolaurate) which is sold as Tween® 20 (Sigma Laboratories, St. Louis, Mo., USA), and .alpha.-[4-(1,1,3,3,-Tetramethylbutyl)phenyl],omega.-hydroxypoly(oxy-1,2-ethanediyl) where the number of ethoxy groups average 10, sold as Triton® X-100 (Rohm and Haas).

Aqueous solutions, dispersions or emulsions may be prepared by dissolving a suitable active inhibitor in water or an organic solvent which may, if desired, contain one or more wetting, dispersing, or emulsifying agents and then, in the case when organic solvents are used, adding the mixture so obtained to water which may, if desired, also contain one or more wetting, dispersing or emulsifying agents. Suitable organic solvents are ethylene dichloride, isopropyl alcohol, propylene glycol, diacetone alcohol, toluene, mineral oil, kerosene, methyl napthalene, xylenes and trichloroethylene.

Inhibitors which are to be used in the form of aqueous solutions, dispersions or emulsions are generally supplied in the form of a concentrate containing a high proportion of the inhibitor, and the concentrate is then diluted with water before use. These concentrates are usually required to withstand storage for prolonged periods and after such storage, to be capable of dilution with water in order to form aqueous preparations which remain homogeneous for a sufficient time to enable them to be applied by conventional spray equipment. In general, concentrates may conveniently contain from 10-60% by weight of a suitable inhibitor or inhibitors. Dilute preparations ready for use may contain varying amounts of the active inhibitor or inhibitors, depending upon the purpose for which they are to be used, and a dilute preparation containing between 0.01 and 10.0% and preferably 0.01 and 1%, by weight of active inhibitor or inhibitors may normally be used.

In carrying out the process of the invention, the amount of suitable inhibitor and herbicide to be applied to reduce or ablate MHR in plants will depend upon a number of factors, for example the particular formulation selected for use, whether the compound(s) is (are) to be applied for foliage or root uptake, the herbicide that is used, and the identity of the plant species involved. However, in general, an application rate of from 0.01 to 100 kg per hectare is suitable, while an application rate of 0.1 to 10 kg per hectare is preferred for most purposes. In all cases routine tests can be used to determine the best rate of application of a specific formulation for any specific purpose for which it is suitable.

The teaching of all references cited herein is incorporated in its entirety into the present description.

### DETAILED DESCRIPTION OF THE INVENTION

There now follow non-limiting examples and figures illustrating the invention.

### LEGEND TO FIGURES

FIG. 1 GSTs in MHR black-grass. (A) 2D-gel electrophoresis of hydrophobic protein fraction from WT and Peldon plants, with polypeptides corresponding to AmGSTF1 arrowed. (B) Western blot of extracts from WT and Peldon plants using an anti-GSTL-serum (C) The effect of 0.1 mM inhibitors on the activity of recombinant black-grass AmGSTF1-1 (100%=640 nkat.mg⁻¹ protein), AmGSTL (100%=83 nkat.mg⁻¹), AmGSTU1 (100%=685 nkat.mg⁻¹) with activities determined using the assays described in table 1.

FIG. 2 HPLC analysis of flavonoid metabolites in the foliage of Arabidopsis (A) WT plants and (B) Amgstfl over-expressors (line 12). Flavonoids showing altered accumulation were identified by HPLC-MS with reference to published data (S17). Compound 1=Kaempferol-7-O-[rhamnosyl-glucosyl-rhamnoside] (M−H)⁻=739; compound 2=cyanidin-3-O-[2-0(2-O-(sinapoyl)-xylosyl)-6-O-(4-O-(β-D-glucosyl)-p-coumaryl-β-D-glucosyl] 5-O-[6-O-(malonyl) β-D-glucoside] (M−H)⁻=1341; compound 3 Kaempferol-7-O-[rhamnosyl-rhamnoside] (M−H)⁻=577 4—Unidentified flavonol glycoside (M−H)⁻=679.

FIG. 3. Effect of transgenic over-expression of AmGSTF1 in Arabidopsis. (A) Amgstfl over-expressors (lines 8, 12) and vector-only controls germinated and grown on agar containing 10 μM herbicides for 30 days. (B) Activities of detoxifying enzymes (substrates in parentheses) and antioxidant contents. (C) Western blot of leaf proteins from vector-only control and lines 8 and 12 probed with maize anti-GSTF-serum.

### EXPERIMENTAL SECTION

### Materials and Methods

Plant Experiments

Seed of the black-grass MHR populations ‘Peldon’ and Spain and from an herbicide-susceptible wild-type (WT) line were obtained from Herbiseed, Twyford, UK. Plants were grown as described previously (1) under two lighting intensity regimes (low=100 μE.m⁻².s⁻¹ or high=220 μE.m⁻².s⁻¹). For biochemical studies, plants were harvested after 30 days (2 to 3 leaf stage), weighed and frozen in liquid nitrogen. For spray trials, herbicides were dissolved in acetone and then diluted (1:100) in 0.1% v/v Tween 20 and applied to 14-day-old plants with a hand-held sprayer at doses equivalent to field rates as expressed as grams active ingredient applied per Hectare (chlortoluron=500 g a.i.ha⁻¹, fenoxaprop ethyl=85 g a.i.ha⁻¹). For studies with inhibitors, plants were pre-treated with chemicals formulated as described for the herbicides, at rates equivalent to 80 g a.i. ha⁻¹.

Arabidopsis thaliana (Columbia) plants were grown and maintained as described previously (2) For transgenic studies, AmGSTF1 was cloned into the XhoI and KpnI sites of the vector pRT107 after introducing the respective restriction sites by PCR, with a 35S promoter driving construct expression and a polyadenylation signal added (2). After cloning into the binary vector pCAMBIA 3300 (CAMBIA, Canberra, Australia), the plasmid was electroporated into Agrobacterium tumefaciens and used to transform Arabidopsis by the floral dip method (3). Homozygote lines were selected after spraying with gluphosinate ammonium (Basta) as described (S2) and analysed for AmGSTF1 expression by western blotting using an anti-GSTF-serum (1). Seed from two lines of Amgstfl-transformants showing intermediate (line 8) and high levels (line 12) of transgene expression were selected for further study. Plants were either grown in growth rooms (irradiance 85 μE.m⁻².s⁻¹), or in the glass-house (up to 1500 μE.m⁻².s⁻¹), and harvested for analysis when 30 days old. Controls were transformed with pCAMBIA 3300 alone. Seeds from lines 8, 12 and controls were germinated on agar containing 2 μM or 10 μM herbicides. Plants were maintained in growth rooms for 30 days and assessed for phytotoxic damage.

Enzyme and Metabolite Assays

All extractions were carried out on ice at 5 v/w of buffer using a pestle and mortar, with samples clarified by centrifugation (10,000 g, 10 min, 4° C.) and protein contents determined using dye binding reagent (Biorad) with γ-globulin as the reference. Enzymes were isolated and assayed as crude extracts at 30° C. using the published methods given in Table 1, with non-enzymic reactions corrected for by using boiled protein controls. Glutathione and hydroxymethylglutathione (4), were quantified in both reduced and oxidised forms by HPLC (Table 1), while ascorbic acid content was determined enzymically (Table 1). Phenolic metabolites were solvent extracted from plant foliage and resolved by HPLC with the eluant analysed by photodiode array detection (PDA) and time-of-flight mass spectrometry following ionization by electrospray ionization time-of-flight mass spectrometry (ESI ToF MS) as described (5). The major flavonoid present in black-grass was purified from WT shoots by preparative HPLC and incubated with 6% v/v conc HCl for 1 h at 95° C., then re-analysed by HPLC ESI ToF MS as above. Sugars released in the hydrolysate were identified by co-chromatography with reference standards on a Carbo-Pak PA-100 HPLC column (4 mm×250 mm, Dionex) coupled with electrochemical detection using a mobile phase of 100 mM NaOH.

Proteomic Analysis

Crude protein extracts were precipitated with ammonium sulfate (40%-80% saturation) and the proteins applied to a phenyl Sepharose column (6). The hydrophobic proteins eluted with 50 mM potassium phosphate were desalted and precipitated with acetone (5 v/v). Proteins were analysed by two-dimensional gel electrophoresis pellets using pH 4-7 isoelectric focusing strips in the first dimension followed by SDS-PAGE on large format gels (7). Polypeptides of interest were identified by staining in Sypro Ruby prior to excision, digestion with trypsin and HPLC MS-MS sequencing (6). Two dimensional, differential, gel electrophoresis of Peldon and WT foliage using fluorescent dyes was carried out on total and hydrophobic protein extracts by the Cambridge Proteomics Centre (http://www.bio.cam.ac.uk/proteomics/).

Analysis of GST Expression

The expression of specific classes of GSTs was monitored by SDS-PAGE and western blotting using antisera raised against maize GSTFs and GSTUs (1) and wheat GSTLs (8). Quantitative PCR was used to determine the abundance of GST transcripts (9). Based on a normalisation with actin (relative abundance=1) values in the control plants were AtGSTL1 (0.0021±0.0002), AtDHAR3 (1.3±0.1) and AtGSTU19 (1.8±0.2). In the Amgstfl-over-expressing lines AtGSTL1 transcript abundance was doubled (0.0047±0.0003), while Atdhar3 and Atgstul19 were unaffected.

Cloning and Expression of AmGSTL1 from Black-Grass

Alignment of DNA sequences encoding GSTLs from wheat (accession Y17386), maize (accession X58573) and rice (accession AF237487) directed the design of oligonucleotides for the initial amplification of a partial sequence of AmGSTL1 prepared from black-grass cDNA (1). Amplifications were performed using the primers AmGSTLF1 (5′ atggccgcagctgcagca 3′), which contained the start codon, together with primers to conserved internal regions, namely AmGSTLF2, (5′ ggtgccttccctggagcacgac 3′ (SEQ ID No. 1)), AmGSTLR1 (5′ gtcgtgctccagggaagg 3′(SEQ ID No. 2)) and AmGSTLR2, (5′ ccaagctaaattggccaaggaagaa 3′ (SEQ ID No. 3)). Amplification products derived from PCR using AmGSTLF1 with the reverse primer generated products, were cloned, sequenced and confirmed to be GSTL-like. The 3′ end of the GSTL sequence was then obtained by 3′ RACE, using AmGSTLF1 and AmGSTLF2 in conjunction with an oligo (dT)-containing primer. The full-length black-grass lambda sequence was then amplified, with the addition of 5′ Nde1 and 3′ Xho 1 sites to allow cloning into pET 24. After confirming its identity, the His-tagged recombinant protein was expressed and affinity purified from E. coli and determined to be active as a thiol transferase (83 nkat.mg⁻¹ pure protein) when assayed with hydroxyethyldisulfide (9).

TABLE 1
methods used to assay enzymes and analyse metabolites

| Enzyme or metabolite | Substrate or analyte | Reference |
|---|---|---|
| Glutathione transferase | 1-chloro-2,4- | 4 |
| | dinitrobenzene (CDNB), | |
| | alachlor and atrazine | |
| Glutathione peroxidase | Cumene hydroperoxide | 1 |
| Thioltransferase | Hydroxyethyldisulfide | 9 |
| Catalase | Hydrogen peroxide | 10 |
| Peroxidase | Ascorbic acid | 11 |
| | Guiacol | 12 |
| Superoxide dismutase | Superoxide | 13 |
| Glutathione reductase | Glutathione (oxidised) | 14 |
| Antioxidant | Glutathione and | 4 and 15 |
| | hydroxymethylglutathione | |
| | (oxidised and reduced) | |
| Antioxidant | Ascorbic acid | 16 |

TABLE 2
Levels of antioxidant enzymes and associated metabolites in
MHR black grass or Amgstf1-expressing Arabidopsis plants
(lines 8 and 12) as compared with the respective controls.
All results are expressed as means +/− SE
(n = 3)

| | Concentration (nmol · g⁻¹ fresh weight) | | |
|---|---|---|---|
| Black Grass | WT | Peldon | Spain |
| Ascorbate | 270 ± 15 | 240 ± 15 | 315 ± 30 |
| | Enzyme activity (nkat⁻¹ fresh weight) | | |
| Ascorbate peroxidase | 2.3 ± 0.1 | 2.8 ± 0 | 2.3 ± 0.2 |
| Guiacol peroxidase | 150 ± 19 | 140 ± 11 | 149 ± 22 |
| Superoxide dismutase | 40.2 ± 0.8 | 38.2 ± 1.6 | 37.5 ± 2.0 |
| (% superoxide | | | |
| remaining in assay vs | | | |
| no enzyme control | | | |
| Arabidopsis | | | |
| | Control | Line 8 | Line 12 |
| Glutathione reductase | 0.434 ± 0 | 0.458 ± 0.009 | 0.406 ± 10 |
| Thiol transferase | 0.029 ± 0.008 | 0.088 ± 0.012 | 0.097 ± 0.009 |
| Catalase | 1949 ± 33 | 1725 ± 163 | 1718 ± 264 |

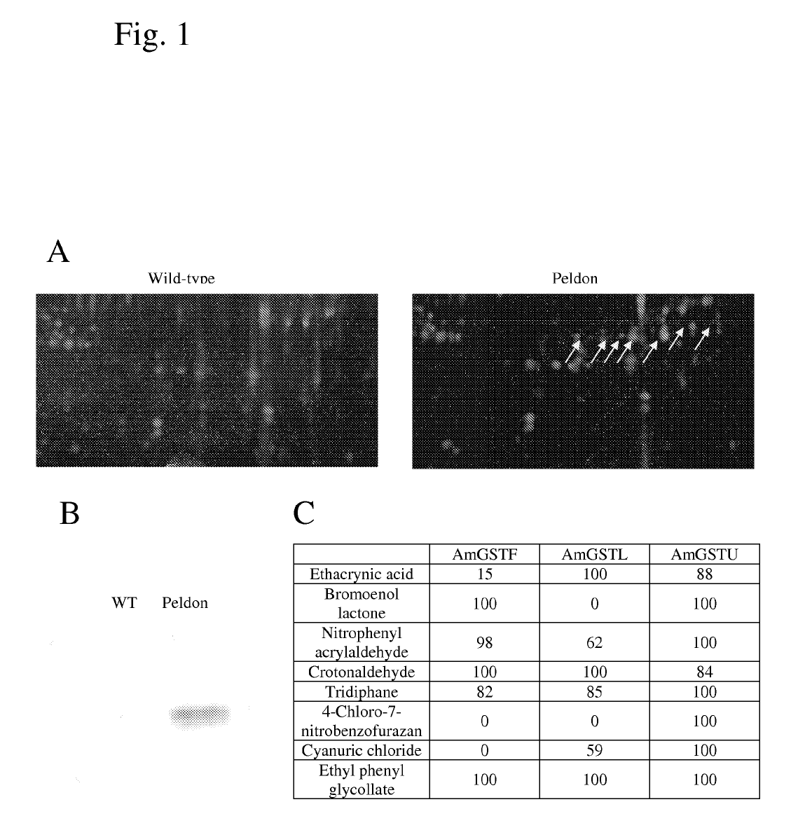

To investigate the biochemical basis of MHR and possible roles for AmGSTF1-1, the resistant Peldon (27) and Spanish (24) populations and a WT line were subjected to metabolic profiling and enzyme assay screens (34). As compared with WTs, when aqueous extracts were analysed for antioxidants, both MHR lines contained elevated concentrations of the tripeptide glutathione and its derivative hydroxymethylglutathione (29), but identical levels of ascorbic acid (Table 1 and Table 2). These changes did not affect the ratio of oxidised: reduced thiols (redox ratio), but were associated with an enhancement in the activities of catalase and five glutathione-dependent enzymes (Table 1). In contrast, ascorbate peroxidase, guiacol peroxidase and superoxide dismutase were unaffected (Table 2). Of the glutathione-dependent enzymes, thioltransferase activity was particularly enhanced in Peldon plants. While this increase was partly explained by the induction of dehydroascorbate reductases, which can also catalyse thiol transfer (33), it was also clear that additional glutathione-dependent enzymes with this activity were present. Plant lambda (L) class GSTs have been shown to be highly active thiol transferases (33). Western blotting of protein extracts with an antibody raised to a GSTL from wheat (35) identified an immunoreactive 27 kDa polypeptide in Peldon plants which was absent in the WTs. Subsequent cloning of the black-grass AmGSTL1 confirmed this identification and the activity of the protein as a thioltransferase (34). Plants were then solvent-extracted and analysed by LC-MS (FIG. 1). As compared with WTs, the foliage of MHR plants contained more anthocyanin pigments and twice the amount of the major flavonoid, tentatively identified as apigenin-6-C-(2″-O-arabinosyl)-glucoside (Table 3).

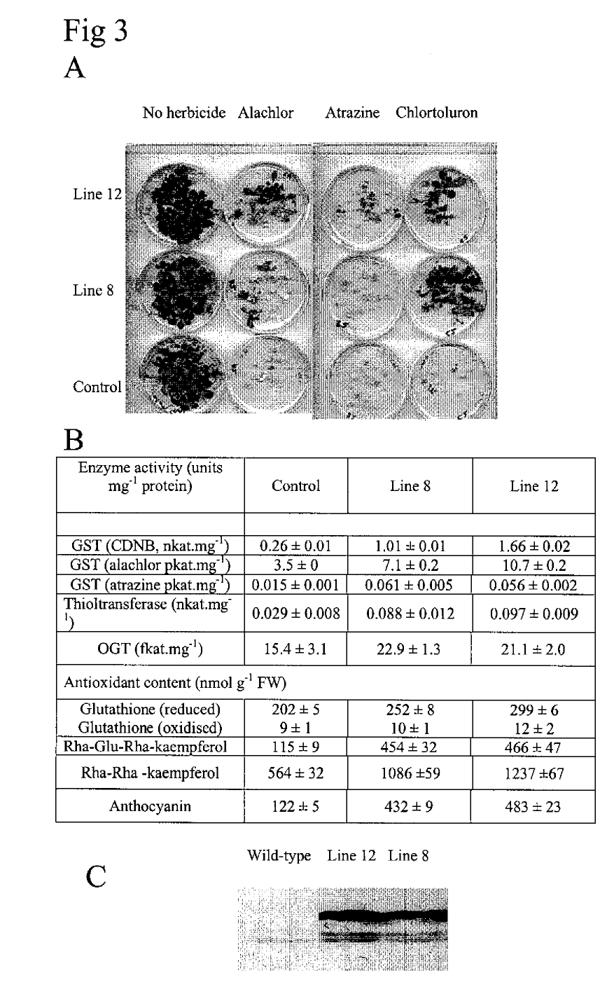

The changes in the proteome in black-grass foliage underlying associated with MHR were then investigated by two-dimensional differential gel electrophoresis following clean-up by hydrophobic interaction chromatography (34). The proteome of Peldon and WT plants were virtually identical, except for 9 polypeptides which were strongly up-regulated in the MHR plants. Seven of these polypeptides had molecular masses (28 kDa) characteristic of GST subunits. When analysed by MALDI ToF MS after digestion with trypsin, all seven gave identical peptide fingerprints, with tandem MS of a 1038 Da fragment identifying the sequence VFGPAMSTNV. This identified all 7 up-regulated polypeptides as AmGSTF1 subunits. This appears to be due to multiple genes encoding variants of AmGSTF1 in Peldon (32). Our results demonstrated that the up-regulation in expression of AmGSTF1 polypeptides was the dominant change associated with the proteome of MHR in black-grass. To investigate the role of this protein, AmGSTF1-1 was constitutively over-expressed in the model plant Arabidopsis thaliana (34) Two independent lines (8 and 12) of homozygous transformants were screened for over-expression of AmGSTF1 by western blotting using an anti-GSTF-serum. These studies confirmed transgene expression, together with the accumulation of immunoreactive Arabidopsis AtGSTFs which were present at low concentration in the vector-only controls. When GST and GPOX activities were determined in the Amgstfl-over-expressors (FIG. 3), the observed increases in atrazine-conjugating activity were found to be due to increased expression of AtGSTs and not AmGSTF1, with this enzyme having no detectable activity toward this substrate. AmGSTF1 expression also caused an enhancement in other detoxifying enzymes in Arabidopsis, notably OGT activity (FIG. 3). While the over-expressors were indistinguishable from controls when grown in growth-rooms, when transferred to glasshouses exposed to full sunlight they became visibly pigmented due to the accumulation of anthocyanins. LC-MS analysis of phenolic metabolites showed 3-fold to 4-fold increases in 2 major anthocyanins and a doubling in the content of the major flavonoids (FIG. 3). Upon analysis for antioxidants and associated enzymes (FIG. 3 table 2), glutathione was found to be modestly enhanced in the over-expressing lines without any disturbance of the redox ratio. Quantitative PCR showed that AmGSTF1 expression doubled the expression of Arabidopsis GSTLs, but had no effect on dehydroascorbate reductases or the tau class enzyme AtGSTU19 (33). Similarly, enzyme assays also showed that other enzymes of antioxidant metabolism which were up-regulated by MHR in black-grass were unaffected by Amgstfl-expression in Arabidopsis (table 2). These results demonstrated that the ectopic expression of AmGSTF1 had selectively replicated part of the MHR phenotype, by enhancing detoxification enzymes, glutathione and flavonoid metabolism. To determine how these changes had affected resistance, Amgstf-expressors were germinated on agar plates containing herbicides with differing modes of action (FIG. 3). The transgenics showed enhanced resistance toward the cell division inhibitor alachlor (a chloroacetanilide) and the PSII inhibitors atrazine (chloro-s-triazine) and chlortoluron (phenylurea). These results showed that expression of AmGSTF1-1 in Arabidopsis conferred tolerance to three distinct classes of herbicide, acting on two target sites. Tolerance to atrazine and alachlor could be partially explained by the direct enhancement in GST-mediated conjugation, and hence detoxification (Table 3). In contrast, resistance to chlortoluron could not be due to enhanced glutathionylation, as this herbicide is inactivated by CYPs and OGTs (26).

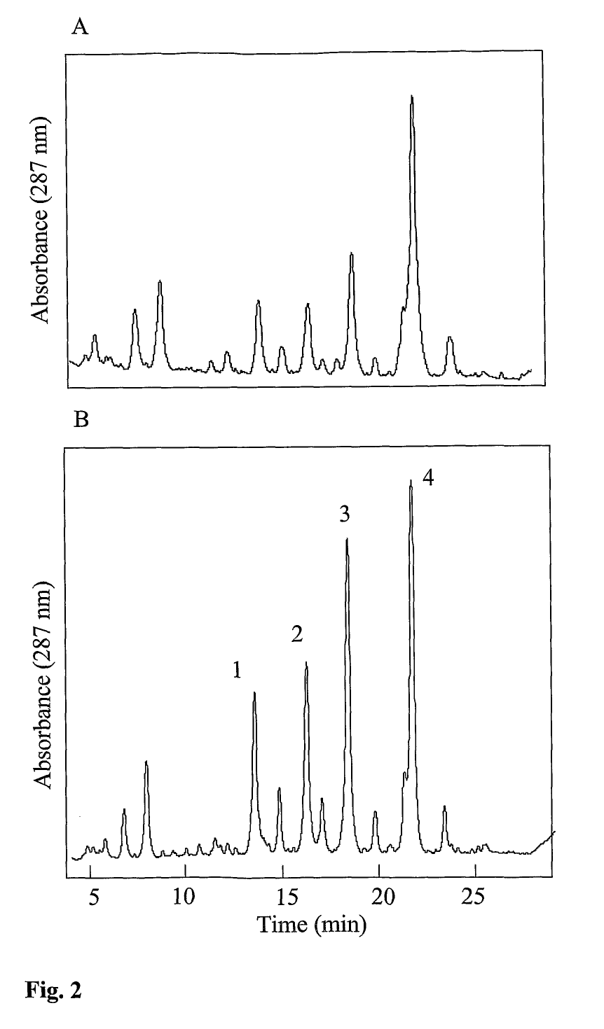

Cumulatively, these experiments showed that when expressed in Arabidopsis, AmGSTF1 orchestrated a series of changes in primary and secondary metabolism, including the enhancement of two distinct pathways of xenobiotic detoxification, which resulted in resistance to multiple herbicides. The identification of such a regulator of MHR, suggested that it would be possible to use chemical intervention to suppress the associated phenotype and restore sensitivity to herbicides in black-grass. Because of their importance in detoxifying chemotherapeutic agents used in cancer therapy, GSTs are a well-recognised target for medicinal chemistry, with a range of inhibitors selective toward different enzymes having been developed (37) Eight different inhibitor chemistries were tested for their ability to disrupt the activity of the black-grass enzymes AmGSTF1, AmGSTL and the herbicide-detoxifying tau (U) class AmGSTU1 (32) AmGSTF1 and AmGSTL1 were totally inhibited by 4-chloro-7-nitro-2,1,3-benzoxadiazole (CNBD) (FIG. 2). Bromoenol lactone (BEL) totally inhibited AmGSTL1, but not AmGSTF1. Based on this differential inhibition of two GSTs which were selectively up-regulated in Peldon, the effect of CNBD and BEL on MHR in black-grass was determined. When applied 48 h prior to an application of chlortoluron, or fenoxaprop ethyl, CNBD but not BEL, reduced resistance to both types of herbicides. Treatment with CNBD also reduced the flavonoid content in MHR Peldon plants to WT levels (FIG. 2).

The results of the transgenesis studies in Arabidopsis and selective inhibitor trials have identified a central role for AmGSTF1 in MHR and a potential mechanism for restoring chemical control. GSTs have long been implicated in tolerance mechanisms to drugs and pesticides due to their well-studied role in conjugating xenobiotics with glutathione, thereby detoxifying them (26, 38). GSTs are also known to exert broad-ranging antioxidant protection to a range of biotic and abiotic stresses in animals, plants and microbes (39, 40). In the case of AmGSTF1 we had previously proposed that this enzyme could counter herbicide action by acting as a GPOX, thereby detoxifying cytotoxic lipid oxidation products formed as a secondary consequence of chemical action (32). The current results demonstrate that AmGSTF1 exerts a much more profound protective effect than previously thought, acting as a causative agent of MHR rather than being part of the protective response. While the mechanisms by which AmGSTF1 co-ordinates signalling events leading to MHR are currently unknown, there are two potential clues arising from the signalling roles of GSTs in animals, which may be relevant. Several mammalian GSTs are known to bind and hence inactivate a c-JUN N-terminal kinase (JNK), which regulates apoptosis and responses to oxidative stress (41) These GSTs attenuate the responsiveness of JNK to oxidative stress and interestingly, thioether derivatives of 2, 1,3-benzoxadiazoles, structurally related to CNBD, have been shown to selectively interfere with this interaction and promote apoptosis (42). Another clue may lie in the unusual antioxidant activity of AmGSTF1-1. The lipid oxidation product, 2-hydroxynonenal (HNE) is known to modulate apoptosis as well as cell differentiation and growth in mammalian cells and it has been proposed that GSTs control this activity (43). Thus, AmGSTF1 would regulate the supply of the hydroperoxide precursors of HNE (32), thereby modulating oxidative stress signalling leading to MHR. The regulatory role of AmGSTF1 in herbicide resistance is currently under investigation. Importantly, the identification of chemical agents which disrupt its signalling function provide both a valuable new research tool to study MHR in wild grasses as well as leads for new agrochemicals to counteract this threat to sustainable arable crop protection.

TABLE 3
levels of antioxidants, flavonoids and redox enzymes in the
foliage of WT and MHR (Peldon, Spain) lines of black-grass.
Values represent means of triplicate determinations ± SE
(n = 3).

| | (nmol · g⁻¹ fresh weight) | | |
|---|---|---|---|
| Antioxidant | Wild-type | Peldon | Spain |
| Glutathione (reduced) | 82 ± 1 | 229 ± 2 | 243 ± 6 |
| Glutathione (oxidised) | 3 ± 1 | 8 ± 2 | 10 ± 4 |
| Hydroxy- | 75 ± 4 | 90 ± 2 | 105 ± 4 |
| methylglutathione | | | |
| (reduced) | | | |
| Hydroxy- | 3 ± 1 | 5 ± 2 | 7 ± 2 |
| methylglutathione | | | |
| (oxidised) | | | |
| Apigenin-6-C-(2″O- | 239 ± 18 | 450 ± 19 | 435 ± 11 |
| arabinosyl) glucoside | | | |
| Anthocyanin | 29 ± 5 | 58 ± 9 | 77 ± 9 |
| | Activity (nkat · mg⁻¹ fresh weight) | | |
| Antioxidant enzymes | Wild-type | Peldon | Spain |
| GST (CDNB) | 1.00 ± 0.05 | 2.41 ± 0.1 | 1.95 ± 0 |
| GPOX | 0.02 ± 0 | 0.06 ± 0.003 | 0.04 ± 0.001 |
| Glutathione reductase | 0.717 ± 0.027 | 1.227 ± 0.034 | 0.957 ± 0.023 |
| Thiol transferase | 0.062 ± 0.009 | 0.274 ± 0.011 | 0.107 ± 0.010 |
| Dehydroascorbate | 0.269 ± 30 | 0.961 ± 28 | 0.848 ± 28 |
| reductase | | | |
| Catalase | 1503 ± 30 | 2819 ± 112 | 2653 ± 176 |

### Synthesis and Screening of Chemical Derivatives

Following the identification of the 4-chloro-7-nitro-2,1,3-benzoxadiazole as an inhibitor of MHR in black-grass, the following derivatives were prepared or obtained:
Compound i) available from Aldrich Chemicals
Compound ii) available from New Horizons Laboratories
Compound iii) available from Maybridge Chemicals
Compound iv) synthesized as described herein
Compound v) synthesized as described herein
Compound vi) available from Fisher Scientific, UK
Compound vii) available from Scientific Exchange Inc.
Compound viii) available from Alfa Aesar
Compound ix) synthesized as described herein
Compound x) synthesized as described herein
Compound xi) synthesized as described herein
Compound xii) synthesized as described herein
Compound xiii) synthesized as described herein
Compound xiv) synthesized as described herein
Compound xv) synthesized as described herein
Compound xvi)* synthesized as described herein
Compound xvii)* synthesized as described herein
Compound xviii)* synthesized as described herein
Compound xix)* synthesized as described herein
Compound xx)* synthesized as described herein
Compound xxi) synthesized as described herein
Compound xxii) synthesized as described herein
Compound xxiii) synthesized as described herein
Compound xxiv)* synthesized as described herein
Compound xxv) synthesized as described herein
Compound xxvi) synthesized as described herein
Compound xxvii)* synthesized as described herein
Compound xxviii) synthesized as described herein
Compound xxix) synthesized as described herein
Compound xxx) available from Aurora screening library
Compound xxxi)* synthesized as described herein
Compound xxxii) synthesized according to the methodology described in Analytica Chimica Acta 344 (1997) 227-232
Compound xxxiii) synthesized as described herein
Compound xxxiv) synthesized according to the teaching of WO2000/076972
Compound xxxv) available from the Maybridge Chemicals
Compound xxxvi) available from Fluorochem
Compound xxxvii) available from Princeton
Compound xxxviii) available from Maybridge Chemicals
Compound xxxix) available from Scientific Exchange Product List
Compound xl) available from TimTec Stock Library
Compound xli) available from Chembridge Screening Library
Compound xlii) available from Ryan Scientific Inc
Compound xliii) available from Ryan Scientific Inc
Compound xliv) available from Ryan Scientific Inc
Compound xlv) available from Sci. Exchange Product List
Compound xlvi) available from Ryan Scientific Inc
Compound xlvii) available from Aurora
Compound xlviii) synthesized as described herein
Compound xlix) synthesized as described in Zhurnal Obshchei Khimii (1966) 26(7) 1268-74
Compound l) available from Ryan Scientific Inc
Compound li) synthesized as described in Zhurnal Obshchei Khimii (1964) 34(1) 261-72
Compound lii) available from Ambinter Stock Screening Collection
Compound liii) synthesized as described herein
Compound liv) available from TCI laboratory Chemicals
Compound liv) available from Ryan Scientific Inc
Compound lvi) synthesized as described herein
Compound lvii) synthesized as described herein
Compound lviii) available from Ryan Scientific Inc
Compound lvix) available from Ryan Scientific Inc
Compound lx) synthesized as described herein
Compound lxi) available from Ryan Scientific Inc
Compound lxii) available from Ryan Scientific Inc
Compound lxiii) available from Ryan Scientific Inc
Compound lxiv) available from TimTec Building Blocks and Reagents
Compound lxv) synthesized as described herein
Compound lxvi) available from Ryan Scientific Inc
Compound lxvii) available from Alfa Aesar

Selected compounds (indicated as compounds i) to lxvii) herein) were applied to 14 day old MHR black-grass plants at a rate equivalent to 80 g a.i. ha⁻¹. The plants were then treated 48 h later with chlortoluron at 500 g a.i.ha⁻¹ as described previously. After 10 days the plants were scored for phytotoxic injury, with +++=full injury (100%) determined with chlortoluron in the presence of CNBD (compound i) of Formula (I)); ++=approximately 50% damage as compared with CNBD; +=minor but measurable injury. Where tested, the score is shown next to the compound.

### Activities of Above Compounds in Phytotoxicity Screen

| Compound of Formula (I) | Score |
|---|---|
| Compound i) | +++ |
| Compound ii) | ++ |
| Compound iii) | +++ |
| Compound iv)* | +++ |
| Compound v)* | + |
| Compound vi) | +++ |
| Compound vii) | + |
| Compound viii) | +++ |
| Compound ix) | +++ |
| Compound x) | + |
| Compound xi) | + |
| Compound xii) | + |
| Compound xiii) | +++ |
| Compound xiv) | ++ |
| Compound xv) | ++ |
| Compound xvi)* | ++ |
| Compound xvii)* | ++ |
| Compound xviii)* | ++ |
| Compound xix)* | ++ |
| Compound xx)* | + |
| Compound xxi) | + |
| Compound xxii) | + |
| Compound xxiii) | +/++ |
| Compound xxiv)* | + |
| Compound xxv) | +++ |
| Compound xxvi) | +++ |
| Compound xxvii)* | +++ |
| Compound xxviii) | + |
| Compound xxix) | +++ |
| Compound xxx) | + |
| Compound xxxi)* | +++ |
| Compound xxxii) | + |
| Compound xxxiii) | + |
| Compound xxxiv) | + |
| Compound xxxv) | ++ |
| Compound xxxvi) | −/+ |
| Compound xxxvii) | −/+ |
| Compound xxxviii) | −/+ |
| Compound xxxix) | +/++ |
| Compound xl) | ++ |
| Compound xli) | + |
| Compound xlii) | + |
| Compound xliii) | + |
| Compound xliv) | + |
| Compound xlv) | + |
| Compound xlvi) | +/++ |
| Compound xlvii) | ++ |
| Compound xlviii) | ++/+++ |
| Compound xlix) | + |
| Compound l) | +/++ |
| Compound li) | + |
| Compound lii) | −/+ |
| Compound liii) | +++ |
| Compound liv) | ++ |
| Compound lv) | ++/+++ |
| Compound lvi) | + |
| Compound lvii) | + |
| Compound lviii) | + |
| Compound lvix) | + |
| Compound lx) | ++ |
| Compound lxi) | + |
| Compound lxii) | ++ |
| Compound lxiii) | ++/+++ |
| Compound lxiv) | −/+ |
| Compound lxv) | + |
| Compound lxvi) | + |
| Compound lxviiδ) | + |

*Novel compounds

### Preamble to Synthesis Section

### Synthesis of Compounds

The halogenated derivatives are all known compounds (Cl and F commercially available)—the other two specific compounds shown are already described in the literature—preparation of all the other derivatives follows from the halo (chloro) analogues following well established literature methods (see R. M. Paton 1,2,5-oxadiazoles in Science of Synthesis, Volume 13 Chapter 7 p185, Thieme, Stuttgart).

### Synthesis of New Compounds

The 7-halo derivatives are prepared by standard condensation reactions between amines or alcohols and the appropriate sulfonyl chloride recognisable by those skilled in the art of organic chemical synthesis—eg see following procedures and data for the two new compounds iv) and v). Further variation of substituents is achieved following analogous procedures as used for the nitro analogues as outlined above.

### 4-(Methylsulfonamido)-7-chloro-2,1,3-benzoxadiazole (Compound iv)

To a solution of 4-chloro-7-chlorosulfonyl-2,1,3-benzoxadiazole (obtainable from Aldrich Chemicals) (0.127 g, 0.50 mmol) in anhydrous acetonitrile (6 mL) was added a solution of 40% wt methylamine in water (0.052 mL, 0.60 mmol) followed immediately by triethylamine (0.084 mL, 0.60 mmol) under argon, with stirring. The reaction was stirred at room temperature for 10 min. before removing the solvent in vacuo. Purification by flash column chromatography on silica gel (PE 40-60:EtOAc, 8:2) gave the title product as a white solid (0.046 g, 0.38 mmol, 37%). ¹H NMR (500 MHz, CDCl₃): δ 8.01 (1H, d, J=7.3, H-5), 7.57 (1H, d, J=7.3, H-6), 5.06 (1H, br q, J=5.2, NH), 2.75 (3H, d, J=5.2, CH₃); ¹³C NMR (125 MHz, CDCl₃): δ 148.8 (C7a), 144.9 (C3a), 134.1 (C5), 129.1 (C6), 127.9 (C7), 126.9 (C4), 29.4 (CH₃).

### 4-(4′-azidobutylsulfonamido)-7-chloro-2,1,3-benzoxadiazole (Compound v)

To a solution of 4-chloro-7-chlorosulfonyl-2,1,3-benzoxadiazole (obtainable from Aldrich Chemicals) (0.127 g, 0.50 mmol) in anhydrous acetonitrile (4 mL) was added a solution of 1-azido-4-aminobutane (0.068 g, 0.60 mmol) in anhydrous acetonitrile (2 mL) followed immediately by triethylamine (0.084 mL, 0.60 mmol) under argon, with stirring. The reaction was stirred at room temperature for 10 min. before removing the solvent in vacuo. Purification by flash column chromatography on silica gel (PE 40-60:EtOAc, 7:3) gave the title product as a white solid (0.099 g, 0.30 mmol, 60%). ¹H NMR (500 MHz, CDCl₃): δ 8.00 (1H, d, J=7.3, H-5), 7.57 (1H, d, J=7.3, H-6), 5.19 (1H, t, J=6.0, NH), 3.30-3.27 (2H, m, CH₂-4′), 3.11-3.06 (2H, m, CH₂-1′), 1.62-1.59 (4H, m, CH₂-2′,3′); ¹³C NMR (125 MHz, CDCl₃): δ 148.8 (C7a), 144.9 (C3a), 133.6 (C5), 129.1 (C6), 127.9 (C7), 127.7 (C4) 50.7 (C4′), 42.9 (C1′), 27.1 (C3′), 25.8 (C2′); Anal. Calcd for C₁₀H₁₁ClN₆O₃S: C, 36.31; H, 3.35; N, 25.41. Found: C, 36.53; H, 3.39; N, 25.33.

For compounds where R¹=CO₂R⁷; CONHR⁴; CONR⁴R⁵; —CN; R²═F, Cl, or Br, NHR⁴, NR⁴R⁵, OR⁴, SR⁴; and R³═H

The carboxylic acid precursors are either commercially available (see below) or may be prepared by oxidation of the known aldehydes (see below) using one of the many known oxidants for such a conversion including KMnO₄, K₂Cr₂O₇, PDC/DMF, Ag₂O, NaClO₂, NaIO₄/RuCl₃, RuO₄.

The carboxy ester and amide derivatives may be prepared by standard condensation reactions between amines or alcohols as employed in the art.

Incorporation of R² substituents to replace the halogen may be achieved following analogous procedures as used for the nitro analogues—see above

Nitriles may be introduced from the respective amino-2,1,3-benzothiadiazole or benzoxadiazole either via Sandmeyer chemistry or by reaction of the bromoheterocycle with Cuprous cyanide in DMF. The nitrile may subsequently be hydrolysed to the carboxylic acid or converted to the thioamide with H₂S (See J. Agric. Food Chem, 1975, 23, 392 and references cited therein)

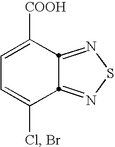

### 4-chloro-2,1,3-benzoxadiazole-7-carboxylic acid

CAN 60, 10670g; 63, 14850f

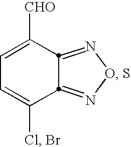

### 4-chloro-2,1,3-benzoxadiazole-7-carboxaldehyde

CAN 92, 94404

### Background Synthesis

The core structures are all well characterised—routes are summarised in Science of Synthesis Volume 13 Chapters 7, 11, 27, Thieme, Stuttgart 2004. This can provide access to the 4 halo derivatives. Nitration or chlorosulfonylation then provides the key intermediates—procedures for these are also documented in this volume

### Atypical Syntheses

4-Bromo-7-nitro-2,1,3-benzoxadiazole/4-Bromo-7-chlorsulfonyl-2,1,3-benzoxadiazole may be prepared by nitration/chlorosulfonylation of the known parent 4-halobenzoxadiazoles (J. Chem Soc B 1971, 2209 and references cited therein).

The benzoselenadiazole and benzothiadiazole series may be generated in a similar fashion from the corresponding 4-halo precursors (for preparation of these see J. Mater. Chem., 2005, 15, 2865; Synth Commun, 1992, 22, 73, J. Heterocyclic Chem. 1970, 7, 629 and references cited therein).

4-chloro-7-formylbenzoxadiazoles may be prepared by a multi-step sequence involving formation of 6-chloranthanil (Tetrahedron Supp 1966, 49), nitration and thermal rearrangement to afford formylbenzofurazan oxide (J. Org. Chem., 1980, 45, 1653; 1977, 42, 897; 1970, 35, 1662) and subsequent reduction with triphenyl phosphine or tributylphosphine.

Key compounds documented in the literature include:

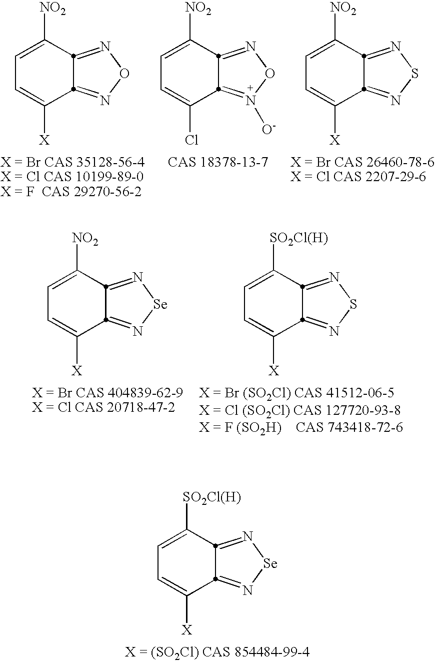

### Experimental Procedures

### General Procedures

All reactions were carried out under an argon atmosphere in glassware dried under high vacuum by a heat-gun unless otherwise stated.

### Solvents

40-60 pet. ether refers to the fraction of petroleum ether boiling between 40 and 60° C. and was redistilled before use. Ether refers to diethyl ether. Solvents were distilled from the following reagents under nitrogen atmosphere: ether and THF (sodium benzophenone ketyl); DCM, xylene and benzene (calcium hydride); chloroform (phosphorus pentoxide) and methanol (sodium methoxide) or obtained from Innovative Technology Solvent Purification System. In cases where mixtures of solvents were utilised, the ratios refer to the volumes used.

### Reagents

Reagents were used as supplied unless otherwise stated. Lithium bromide was made anhydrous by heating at 100° C. at 0.06 mmHg for 3 h. Magnesium bromide was synthesised by addition of 1,2-dibromoethane to an equivalent amount of magnesium in ether. Aldehydes and dienes were distilled, immediately prior to use, from anhydrous calcium sulphate and sodium borohydride, respectively.

### Chromatography

Flash chromatography was carried out using silica gel 40-63μ. Analytical thin layer chromatography (TLC) was performed using precoated glass-backed plates (silica gel 60 F₂₅₄) and visualised by UV radiation at 254 nm, or by staining with phosphomolybdic acid in ethanol or potassium permanganate in water.

### Melting Point

All melting points were determined using a Gallenkamp melting point apparatus and are uncorrected.

### Gas Chromatography

Gas chromatography was carried out on a Hewlett-Packard 5890 Series II fitted with a 25 m column. Detection was by flame ionisation.

### IR Spectroscopy

Infrared spectra were recorded using a Diamond ATR (attenuated total reflection) accessory (Golden Gate) or as a solution in chloroform via transmission IR cells on a Perkin-Elmer FT-IR 1600 spectrometer.

### NMR Spectroscopy

¹H NMR spectra were recorded in CDCl₃ on Varian Mercury 200, Varian Unity-300, Varian VXR-400 or Varian Inova-500 instruments and are reported as follows; chemical shift δ (ppm) (number of protons, multiplicity, coupling constant J (Hz), assignment). Residual protic solvent CHCl₃ (δ_{H}=7.26) was used as the internal reference. ¹³C NMR spectra were recorded at 63 MHz or 126 MHz, using the central resonance of CDCl₃ (δ_{C}=77.0 ppm) as the internal reference. All chemical shifts are quoted in parts per million relative to tetramethylsilane (δ_{H}=0.00 ppm) and coupling constants are given in Hertz to the nearest 0.5 Hz. Assignment of spectra was carried out using COSY, HSQC, HMBC and NOESY experiments.

### Mass Spectroscopy

Gas chromatography-mass spectra (EI) were obtained using a Thermo TRACE mass spectrometer. Electrospray mass spectra (ES) were obtained on a Micromass LCT mass spectrometer. High resolution mass spectra were obtained using a Thermo LTQ mass spectrometer (ES) at the University of Durham, or performed by the EPSRC National Mass Spectrometry Service Centre, University of Wales, Swansea.

### Experimental Details

### 4-Bromo-7-nitrobenzo[c][1,2,5]oxadiazole (JDS096-2)⁵¹ (Compound viii)

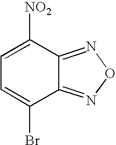

### Stage 1

A solution of 2,6-dibromoaniline (1.0 g, 4.0 mmol) in CHCl₃ (8 ml) was treated with a suspension of m-CPBA (2.1 g, 12.0 mmol) in CHCl₃ (8 ml) and the resulting mixture stirred overnight. After 24 h the solution was diluted with CHCl₃ and washed successively with sat. aq. Na₂S₂O₃, sat. aq. NaHCO₃ and brine. The organic layers were dried over Na₂SO₄, filtered, concentrated and dried in vacuo to afford 1,3-dibromo-2-nitrosobenzene (1.0 g, 100%); the resultant solid was used directly in the next stage.

### Stage 2

1,3-Dibromo-2-nitrosobenzene (1.0 g, 3.8 mmol) was suspended in DMSO (15 ml) and treated with a solution of sodium azide (0.3 g, 4.2 mmol) in DMSO (15 ml) at room temperature. The resultant solution was stirred for 2 h, until nitrogen evolution had ceased, then was warmed to 120° C. for 5 mins. After cooling to room temperature the solution was poured onto crushed ice and the resulting precipitate was filtered and dried in vacuo to afford 4-bromobenzo[c][1,2,5]oxadiazole (0.7 g, 93%); v_{H} (400 MHz, CDCl₃) 7.83 (1H, d, J9, Ar—H), 7.64 (1H, d, J7, Ar—H), 7.30 (1H, dd, J9, 7, Ar—H); the resultant solid (Compound 34) was used directly in the next stage.

### 4-bromobenzo[c][1,2,5]oxadiazole (Compound xxxiv)

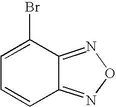

Stage 3

4-Bromobenzo[c][1,2,5]oxadiazole (0.7 g, 3.5 mmol) was redissolved in H₂SO₄ (5 ml) and treated dropwise with a solution of KNO₃ (0.5 g, 4.7 mmol) in 50% H₂SO₄ (3 ml). The resulting solution was heated to 85° C. for 30 mins and then poured onto crushed ice. The aqueous material was then extracted with EtOAc (3×15 ml), dried over Na₂SO₄, filtered, concentrated and dried in vacuo. Flash chromatography (n-hexane, n-hexane/EtOAc [9:1], [4:1]) afforded the title compound as a tan solid (0.47 g, 49%); R_{f} 0.5 (n-hexane/EtOAc 4:1); m.p. 92-94° C.; vₘₐₓ (thin film) 1509, 1443, 1322, 1042, 997, 933, 858, 801, 728, 604 cm⁻¹; δ_{H} (400 MHz, CDCl₃) 8.38 (1H, d, J 8, Ar—H), 8.67 (1H, d, J 8, Ar—H); δ_{C} (126 MHz, CDCl₃) 150.5 (C═N), 142.3 (C═N), 136.2 (ipso-Ar—C), 132.1 (Ar—C), 130.4 (Ar—C), 119.1 (ipso-Ar—C); m/z (EI) 245 ([⁸¹Br]MH⁺, 2%), 243 ([⁷⁹Br]MH⁺), 215 (2%), 213 (2%), 185 (6%); HRMS (EI) Found M⁺, 242.9271 (C₆H₂⁷⁹BrN₃O₃ requires 242.9274).

### 4-(Methylthio)-7-nitrobenzo[c][1,2,5]oxadiazole (JDS094-1)⁵⁰ (Compound 1x)

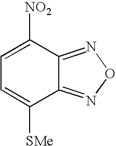

NBD-Cl (0.4 g, 2.0 mmol) was dissolved in ethanol:0.1M sodium phosphate buffer (1:1 v/v, 40 ml). Then sodium methanethiolate (0.17 g, 2.4 mmol) was added and the reaction was stirred for 3 h after which time the resultant precipitate was filtered. The precipitate was then subjected to flash chromatography (CHCl₃) to afford the title compound as a orange solid (0.33 g, 77%); R_{f} 0.4 (CHCl₃); m.p. 118-120° C.; vₘₐₓ (thin film) 1497, 1418, 1308, 1280, 1122, 1044, 957, 847, 733 cm⁻¹; δ_{H} (700 MHz, CDCl₃) 8.43 (1H, d, J 8, Ar—H), 7.10 (1H, d, J 8, Ar—H), 2.77 (3H, s, Ar—SCH₃); δ_{C} (176 MHz, CDCl₃) 149.0 (ipso-Ar—C), 142.5 (C═N), 142.4 (C═N), 132.7 (ipso-Ar—C), 130.6 (Ar—C), 119.5 (Ar—C), 14.7 (Ar—SCH₃); m/z (EI) 211 (M⁺); HRMS (ES⁺) Found MNH₄⁺, 229.0387 (C₇H₉N₄O₃S requires 229.0390).

### 4-Nitro-7-(4-(trifluoromethyl)phenylthio)benzo[c][1,2,5] oxadiazole (JDS0120) (Compound x)

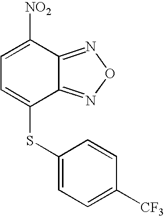

NBD-Cl (0.2 g, 1.0 mmol) was dissolved in ethanol:0.1M sodium phosphate buffer (1:1 v/v, 20 ml). Then 4-(trifluoromethyl)thiophenol (0.17 ml, 1.2 mmol) was added and the reaction was stirred for 2 h, after which time the resultant precipitate was filtered. The precipitate was then washed with water and ethanol to afford the title compound as a yellow solid (0.20 g, 57%); m.p. 134-138° C.; v. (thin film) 1509, 1315, 1169, 1130, 1099, 1050, 952, 835 cm⁻¹; δ_{H} (500 MHz, CDCl₃) 8.30 (1H, d, J 8, Ar—H), 7.85 (4H, s, Ar—H), 6.76 (1H, d, J 8, Ar—H); δ_{C} (126 MHz, CDCl₃) 148.5 (C═N), 142.5 (C═N), 140/4 (ipso-Ar—C), 135.8 (ipso-Ar—C), 133.3 (Ar—CF₃, m), 131.2 (ipso-Ar—C), 130.4 (Ar—C), 127.5 (4Ar—C), 124.5 (ipso-Ar—C), 122.7 (Ar—C); δ_{F} (188 MHz, CDCl₃)-63.5 (3F, s, Ar—CF₃); m/z (EI) 341 (M⁺, 100%), 322 (20%), 272 (50%), 265 (40%), 253 (45%), 242 (43%), 195 (70%), 176 (25%), 157 (35%), 145 (40%), 119 (35%); HRMS (EI) Found M⁺, 341.0080 (C₁₃H₆F₃N₃O₃S requires 341.0076)

### 4-Morpholino-7-nitrobenzo[c][1,2,5]oxadiazole (JDS0123) (Compound xi)

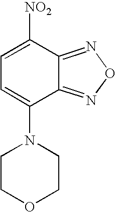

NBD-Cl (0.2 g, 1.0 mmol) was dissolved in ethanol:0.1M sodium phosphate buffer (1:1 v/v, 20 ml). Then morpholine (0.1 ml, 1.2 mmol) was added and the reaction was stirred for 2 h, after which time the resultant precipitate was filtered. The precipitate was then washed with water and ethanol to afford the title compound as a red solid (0.22 g, 90%); m.p. sublimes ° C.; v. (thin film) 2368, 2342, 2240, 1601, 1548, 1482, 1438, 1292, 1259, 1211, 1163, 1115, 1035, 993 cm⁻¹; δ_{H} (500 MHz, CDCl₃) 8.45 (1H, d, J 9, Ar—H), 6.33 (1H, d, J 9, Ar—H), 4.08 (4H, m, (CH₂)₂), 3.96 (4H, m, (CH₂)₂); δ_{C} (126 MHz, CDCl₃) 145.2 (ipso-C), 144.9 (C═N), 144.7 (C═N), 134.9 (Ar—C), 102.6 (Ar—C), 66.3 ((CH₂)₂), 49.4 ((CH₂)₂); m/z (ES⁺) 251 (MH⁺), 523 (2MNa⁺); HRMS (ES⁺) Found MH⁺, 251.0775 (C₁₀H₁₁N₄O₄ requires 251.0775).

### 4-Ethoxy-7-nitrobenzo[c][1,2,5]oxadiazole (JDS082-1)⁴⁸ (Compound xii)

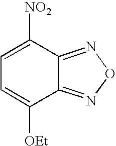

NBD-Cl (0.4 g, 2.0 mmol) was dissolved in ethanol:0.1M sodium phosphate buffer (1:1 v/v, 40 ml). Then ethylene glycol (0.1 ml, 2.2 mmol) was added and the pH adjusted to 7 with 1M aq. NaOH. The reaction was stirred for 3 h then mixed with sat. aq. NH₄Cl (10 ml) and extracted with EtOAc (3×15 ml). The organic layers were dried over MgSO₄, filtered, concentrated and dried in vacuo. Flash chromatography (n-hexane/EtOAc [9:1], [4:1], [7:3]) afforded the title compound as a light brown solid (0.01 g, 2%); R_{f} 0.6 (n-hexane/EtOAc 1:1); δ_{H} (400 MHz, CDCl₃) 8.53 (1H, d, J 8, Ar—H), 6.67 (1H, d, J 8, Ar—H), 4.47 (2H, q, J 7, OCH₂CH₃), 1.63 (3H, t, J 7, OCH₂CH₃); δ_{c} (126 MHz, CDCl₃) 154.8 (ipso-Ar—C), 145.2 (C═N), 143.9 (C═N), 134.2 (Ar—C), 129.5 (ipso-Ar—C), 104.3 (Ar—C), 67.1 (OCH₂CH₃), 14.2 (OCH₂CH₃); m/z (ES⁺) 232 (MNa⁺), 264 (MNa⁺MeOH⁺).

### 7-Nitrobenzo[c][1,2,5]oxadiazol-4-ol (JDS035)⁴⁴ (Compound xiii)

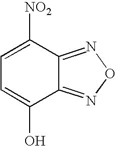

### Stage 1

4-Chloro-7-nitrobenzo[c][1,2,5]oxadiazole (NBD-C1, 1.0 g, 5.0 mmol) was dissolved in MeOH (25 ml) and added dropwise at room temperature via cannula to a freshly made 1N solution of NaOMe (0.23 g Na in 10 ml MeOH). The solution was stirred for 2 h then treated carefully with sat aq. NH₄Cl. The mixture was then extracted with Et₂O (3×20 ml). The organic layers were dried over MgSO₄, filtered, concentrated and dried in vacuo to afford 4-methoxy-7-nitrobenzo[c][1,2,5]oxadiazole as an off white solid (0.76 g, 66%); δ_{H} (400 MHz, CDCl₃) 8.56 (1H, d, J 8, Ar—H), 6.68 (1H, d, J 8, Ar—H), 4.24 (3H, s, OCH₃); the resultant solid was used directly in the next stage.⁴⁵

Stage 2

4-Methoxy-7-nitrobenzo[c][1,2,5]oxadiazole (0.76 g, 3.9 mmol) (Compound 34) was added to hot 1% NaOH (10 ml). The resultant solution was refluxed for 30 min then cooled to room temperature. The aqueous material was then acidified with c. HCl and extracted with Et₂O (3×20 ml). The organic layers were dried over MgSO₄, filtered, concentrated and dried in vacuo to afford the title compound as a brown solid (0.5 g, 71%); R_{f} 0.4 (DCM/MeOH 4:1); v. (thin film) 3260-2788 (broad-OH), 1638, 1560, 1518, 1310, 1084, 1002, 913, 875, 840 cm⁻¹; δ_{H} (200 MHz, CD₃OD) 8.60 (1H, d, J 8,6-H), 6.73 (1H, d, J 8, 5-H). δ_{C} (176 MHz, CDCl₃) 156.4 (C═NO), 146.9 (ipso-Ar—C), 145.7 (ipso-Ar—C), 136.8 (Ar—C), 108.7 (Ar—C); m/z (EI) 181 (M⁺, 45%), 121 (25%), 99 (40%), 80 (80%), 76 (100%), 75 (65%), 71 (50%), 64 (75%), 52 (85%); HRMS (EI) Found M⁺, 181.0119 (C₆H₃N₃O₄ requires 181.0118); Elemental Analysis [Found C, 40.23%; H, 1.75%; N, 23.01% required for C₆H₃N₃O₄: C, 39.79%; H, 1.67%; N, 23.20%]; all data agree with those reported in the literature.⁴⁵

### 3-(7-Nitrobenzo[c][1,2,5] oxadiazol-4-ylthio)propanoic acid (JDS078)⁴⁷ (Compound xiv)

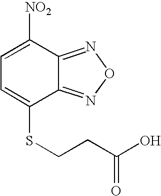

NBD-C1 (0.2 g, 1.0 mmol) was dissolved in ethanol:0.1M sodium phosphate buffer (1:1 v/v, 20 ml). Then 3-mercapto-1-propionic acid (0.1 ml, 1.1 mmol) was added and the pH adjusted to 7 with 1M aq. NaOH. The reaction was stirred for 6 h after which time the resultant precipitate was filtered and washed with water (2×15 ml) and dried to afford the title compound as a light brown solid (0.16 g, 61%); δ_{H} (700 MHz, (CD₃)₂C0) 8.58 (1H, d, J 8, Ar—H), 7.63 (1H, d J 8, Ar—H), 3.64 (2H, t, J 7 SCH₂CH₂COOH), 2.93 (2H, t, J 7, SCH₂CH₂COOH); δ_{c} (175 MHz, (CD₃)₂C0) 171.7 (C═O), 149.8 (C═N), 143.2 (C═N), 139.9 (ipso-Ar—C), 133.3 (ipso-Ar—C), 131.9 (Ar—C), 122.2 (Ar—C), 32.3 (SCH₂CH₂COOH), 26.4 (SCH₂CH₂COOH); m/z (ES) 196 (M⁻-CO₂H, —CH₂CH₂), 268 (M), 536 (2M⁻).

### 6-(7-Nitrobenzo[c][1,2,5] oxadiazol-4-ylthio)hexan-1-ol (JDS081)⁴⁷ (Compound xv)

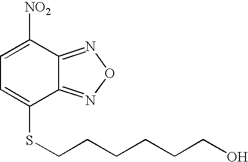

NBD-Cl (0.4 g, 2.0 mmol) was dissolved in ethanol:0.1M sodium phosphate buffer (1:1 v/v, 40 ml). Then 6-mercaptohexan-1-ol (0.3 ml, 2.2 mmol) was added and the pH adjusted to 7 with 1M aq. NaOH. The reaction was stirred for 3 h after which time the resultant precipitate was filtered and washed with water (2×15 ml) and dried to afford the title compound as a light brown solid (0.44 g, 74%); δ_{H} (400 MHz, CDCl₃) 8.40 (1H, d, J 8, Ar—H), 7.14 (1H, d, J 8, Ar—H), 3.67 (2H, t, J 6, CH₂OH), 3.28 (2H, t, J7, CH₂S), 1.88 (2H, q, J7, CH₂CH₂CH₂), 1.63-1.45 (6H, m, (CH₂)₃); δ_{c} (126 MHz, CDCl₃) 149.2 (ipso-Ar—C), 142.5 (C═N), 141.9 (C═N), 130.6 (Ar—C), 120.2 (Ar—C), 62.7 ((CH₂)₆), 32.4 ((CH₂)₆), 31.7 ((CH₂)₆), 28.6 ((CH₂)₆), 27.8 ((CH₂)₆), 25.3 ((CH₂)₆); m/z (ES⁺) 320 (MNa⁺);

### 7-Bromo-N-propylbenzo[c][1,2,5] thiadiazole-4-carboxamide (JDS049-1) (Compound xvi)

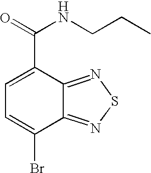

Following the standard procedure outlined hereinabove, 7bromobenzo[c][1,2,5]thiadiazole-4-carboxylic acid (0.06 g, 0.23 mmol) was transformed into the title compound which was isolated as a white solid (0.008 g); R_{f} 0.5 (n-hexane/EtOAc 7:3); m.p. 110-115° C.; vmax(thin film) 3350 (NH), 2955, 2920, 2869, 1636 (C═O), 1520, 1477, 1305, 1261, 1190, 886 cm⁻¹; δH (700 MHz, CDCl₃) 8.97 (1H, bs, NH), 8.49 (1H, d, J 8, Ar—H), 8.00 (1H, d, J 8, Ar—H), 3.56 (2H, q, J 7, NHCH₂CH₂CH₃), 1.75 (2H, sextet, J 7, NHCH₂CH₂CH₃), 1.05 (3H, t, J 7, NHCH₂CH₂CH₃); δC (176 MHz, CDCl₃) 162.6 (C═O), 153.6 (C═N), 151.5 (C═N), 133.5 (Ar—C), 132.3 (Ar—C), 124.6 (ipso-Ar—C), 118.4 (ipso-Ar—C), 41.9 (NHCH₂CH₂CH₃), 22.8 (NHCH₂CH₂CH₃), 11.6 (NHCH₂CH₂CH₃); m/z (ES⁺) 302 ([⁸¹Br]MH⁺), 300 ([⁷⁹Br]MH⁺), 324 ([⁸¹Br]MNa⁺), 322 ([⁷⁹Br]MNa⁺), 625 ([⁸¹Br]2MNa⁺), 623 ([⁷⁹Br,⁸¹Br]MNa⁺), 621 ([⁷⁹Br]2MNa⁺); HRMS (ES⁺) Found MH⁺, 299.98013 (C₁₀H₁₁ON₃⁷⁹BrS requires 299.98007)

### 7-Bromo-N-methylbenzo[c][1,2,5]thiadiazole-4-carboxamide (JDS050) (Compound xvii)

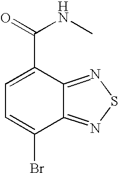

Following the standard procedure outlined herein, 7-bromobenzo[c][1,2,5]thiadiazole-4-carboxylic acid (0.06 g, 0.23 mmol) was transformed into the title compound which was isolated as a white solid (0.012 g, XX %); R_{f} 0.2 (n-hexane/EtOAc 7:3); m.p. 156-160° C.; vmax(thin film) 3330 (NH), 2944, 2896, 2854, 1637 (C═O), 1555, 1520, 1475, 1303, 1264, 1197, 856 cm⁻¹; δH (700 MHz, CDCl₃) 8.91 (1H, bs, NH), 8.49 (1H, d, J 8, Ar—H), 8.00 (1H, d, J 8, Ar—H), 3.15 (3H, d, J 5, NHCH₃); δ_{c} (176 MHz, CDCl₃) 163.3 (C=0), 153.5 (C═N), 151.4 (C═N), 133.5 (Ar—C), 132.2 (Ar—C), 124.4 (ipso-Ar—C), 118.5 (ipso-Ar—C), 26.8 (NHCH₃); m/z (ES⁺) 274 ([⁸¹Br]MH⁺), 272 ([⁷⁹Br]MH⁺), 296 ([⁸¹Br]MNa⁺), 294 ([⁷⁹Br]MNa⁺); HRMS (ES⁺) Found MH⁺, 271.94879 (C₈H₇⁷⁹BrN₃OS requires 271.94877)

### 7-Bromo-N,N-dimethylbenzo[c][1,2,5]thiadiazole-4-carboxamide (JDS051-1) (Compound xviii)

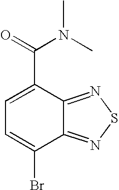

Following the standard procedure outlined herein, 7-bromobenzo[c][1,2,5]thiadiazole-4-carboxylic acid (0.06 g, 0.23 mmol) was transformed into the title compound which was isolated as a colourless oil (0.006 g); R_{f} 0.2 (n-hexane/EtOAc 7:3); vmax(thin film) 2922, 2856, 2809, 1629 (C═O), 1532, 1396, 1141, 876, 841 cm⁻¹; δH (700 MHz, CDCl₃) 7.90 (1H, d, J 7, Ar—H), 7.54 (1H, d, J 7, Ar—H), 3.24 (3H, s, N(CH₃)₂), 2.89 (3H, s, N(CH₃)₂); δC (176 MHz, CDCl₃) 166.9 (C═O), 153.2 (C═N), 151.1 (C═N), 131.8 (Ar—C), 129.6 (ipso-Ar—C), 128.6 (Ar—C), 115.6 (ipso-Ar—C), 38.9 (N(CH₃)₂), 35.2 (N(CH₃)₂); m/z (ES⁺) 287.9 ([⁸¹Br]MH⁺), 285.9 ([⁷⁹Br]MH⁺), 309.9 ([⁸¹Br]MNa⁺), 307.9 ([⁷⁹Br]MNa⁺), 596.8 ([⁸¹Br]2MNa⁺), 594.8 ([^{81.79}Br]2MNa⁺), 592.8 ([⁷⁹Br]2MNa⁺); HRMS (ES⁺) Found MNa⁺, 307.94640 (C₉H₈BrN₃NaOS requires 307.94637)

### Methyl 7-bromobenzo[c][1,2,5]thiadiazole-4-carboxylate (JDS053)(Compound xix)

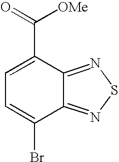

Following the standard procedure outlined herein, 7-bromobenzo[c][1,2,5]thiadiazole-4-carboxylic acid (0.06 g, 0.23 mmol) was transformed into the title compound which was isolated as a white solid (0.011 g); R_{f} 0.7 (n-hexane/EtOAc 7:3); m.p. 122-126° C.; vmax(thin film) 2956, 2928, 2848, 1708 (C=0), 1523, 1329, 1303, 1260, 1194, 1165, 939, 891, 853 cm¹; δH (700 MHz, CDCl₃) 8.25 (1H, d, J 8, Ar—H), 7.95 (1H, d, J 8, Ar—H), 4.07 (3H, s, —COOCH₃); δC (176 MHz, CDCl₃) 164.6 (C═O), 154.1 (C═N), 151.5 (C═N), 133.8 (Ar—C), 131.2 (Ar—C), 122.5 (ipso-Ar—C), 120.5 (ipso-Ar—C), 52.8 (COOCH₃); m/z (ES⁺) 275 ([⁸¹Br]M⁺), 273 ([⁷⁹Br]M⁺), 297 ([⁸¹Br]MNa⁺), 295 ([⁷⁹Br]MNa⁺), 571 ([⁸¹Br]2MNa⁺), 569 ([⁸¹Br,⁷⁹Br]2MNa⁺), 567 ([⁷⁹Br]2MNa⁺); HRMS (ES⁺) Found MH⁺, 272.93264 (C₈H₆⁷⁹BrN₂O₂S requires 272.93279)

### Methyl 4-(7-bromobenzo[c][1,2,5]thiadiazol-4-yl)benzoate (JDS017-1) (Compound xx)

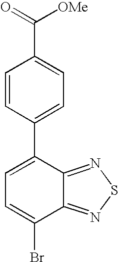

4,7-Dibromobenzo[c][1,2,5]thiadiazole (0.25 g, 0.85 mmol), 4-(methoxycarbonyl)phenylboronic acid (0.15 g, 0.85 mmol), Pd(PPh₃)₄ (0.014 g, 0.01 mmol) and Na₂CO₃ (0.09 g, 0.85 mmol) were weighed into a round bottom flask and dissolved with toluene (1 ml), THF (1 ml) and H₂O (0.2 ml). The solution was then refluxed for 24 h, cooled and poured in to H₂O. The aqueous layer was extracted with Et₂O (3×15 ml). The organic layers were dried over MgSO₄, filtered, concentrated and dried in vacuo. Flash chromatography (n-hexane/DCM [4:1], [6:4], [4:6]) afforded the title compound as a pale yellow solid (0.08 g, 26%); R_{f} 0.3 (n-hexane/DCM 6:4); m.p. 188-192° C.; vmax(thin film) 1732 (C═O), 1608, 1480, 1430, 1317, 1274, 1185, 1151, 1110, 944, 881, 830, 765, 700 cm⁻¹; δH (500 MHz, CDCl₃) 8.20 (1H, d, J 8, Ar—H), 7.98 (1H, d, J 8, Ar—H), 7.96 (1H, d, J 8, Ar—H), 7.64 (1H, d, J 8, Ar—H), 3.97 (3H, s, OCH₃); δC (126 MHz, CDCl₃) 166.7 (C═O), 153.8 (ipso-Ar—C), 152.8 (ipso-Ar—C), 140.9 (ipso-Ar—C), 132.8 (ipso-Ar—C), 132.2 (Ar—C), 130.1 (ipso-Ar—C), 129.9 (Ar—C), 129.1 (Ar—C), 128.8 (Ar—C), 114.2 (ipso-Ar—C), 52.3 (OCH₃); m/z (EI) 350 ([⁸¹Br]M⁺, 100%), 348 ([⁷⁹Br]M⁺, 90%), 319 ([⁸¹Br]M⁺-OCH₃, 90%), 317 ([⁷⁹Br]M⁺-OCH₃, 80%), 291 ([⁸¹Br]M⁺-OCH₃, —C═O, 40%), 289 ([⁷⁹Br]M⁺-OCH₃, —C═O, 35%), 209 (70%); HRMS (EI) Found [⁷⁹Br]M⁺, 347.9560 (C₁₄H₉⁷⁹BrN₂O₂S requires 347.9563) Further elution of the column gave 4,7-dibromobenzo[c][1,2,5]thiadiazole (0.1 g, 50%) and the title compound as a mixture with the di-coupled product (0.05 g, 25%).

### 4-Bromo-7-nitrobenzo[c][1,2,5]thiadiazole (JDS088-1)⁴⁹ (Compound xxi)

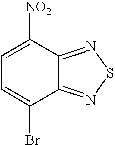

4,7-Dibromobenzo[c][1,2,5]thiadiazole (1.0 g, 3.4 mmol) was suspended in 70% HNO₃ (6 ml). The reaction was then heated to reflux for 2 h until a solution had formed. The reaction mixture was then poured onto ice, warmed back to room temperature and the precipitate that has formed was filtered. Flash chromatography of the solid material (CHCl₃) afforded the title compound as a pale yellow solid (0.06 g, 7%); R_{f} 0.3 (CHCl₃); m.p. 216-218° C.; vmax(thin film) 3051, 1502, 1342, 1314, 1195, 1001, 939, 653, 815, 730, 571 cm⁻¹; δH (700 MHz, CDCl₃) 8.48 (1H, d, J 7, Ar—H), 8.04 (1H, d, J 8, Ar—H); δC (176 MHz, CDCl₃) 154.6 (C═N), 145.8 (C═N), 139.0 (ipso-Ar—C), 130.4 (Ar—C), 127.7 (Ar—C), 123.1 (ipso-Ar—C); m/z (EI) 261 ([⁸¹Br]MH⁺, 70%), 259 ([⁷⁹Br]MH⁺, 65%), 231 (100%), 229 (85%), 203 (45%), 201 (45%); HRMS (EI) Found M⁺, 258.9048 (C₆H₂⁷⁹BrN₃O₂S requires 258.9046).

### 4-Bromo-7-nitrobenzo[c][1,2,5] selenadiazole (JDS095-2) (Compound xxii)

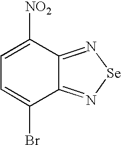

4,7-Dibromobenzo[c][1,2,5]selenadiazole (0.5 g, 1.5 mmol) was suspended in 70% HNO₃ (6 ml) and H₂O (2 ml). The reaction was then heated to reflux for 5 h, cooled to room temperature and filtered. Flash chromatography of the solid material (CHCl₃) afforded the title compound as a pale yellow solid (0.01 g, 3%); R_{f} 0.4 (CHCl₃); m.p. >300° C.; vmax(thin film) 1508, 1471, 1321, 1257, 1090, 992, 920, 855, 814, 766, 727 cm⁻¹; δH (400 MHz, CDCl₃) 8.36 (1H, d, J 8, Ar—H), 7.96 (1H, d, J 8, Ar—H);

### 7-Bromobenzo[c][1,2,5]thiadiazole-4-carboxylic acid (JDS057-1) (Compound xxiii)

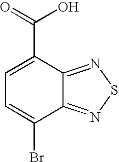

4-Bromo-7-methylbenzo[c][1,2,5]thiadiazole (0.25 g, 1.1 mmol) was dissolved in AcOH (12 ml) and H₂SO₄ (1.7 ml). [CAUTION: Exothermic]. The solution was then treated with chromium (VI) oxide (1.5 g) portionwise and stirred for 30 mins. The reaction was then poured onto ice and allowed to warm to room temperature. The aqueous layer was then extracted with DCM (3×30 ml). The aqueous layer was left overnight and the precipitate filtered and washed with water (3×2 ml) to afford the title compound as a white solid (0.085 g, 30%); vmax(thin film) 3100-3300 (broad —OH), 1682 (C═O), 1680, 1525, 1312, 1289, 1193 cm⁻¹; δH (500 MHz, (CD₃)₂C0) 8.31 (1H, d, J 8, Ar—H), 8.15 (1H, d, J 8, Ar—H), 2.09 (1H, s, CO₂H); δC (126 MHz, (CD₃)₂C0) 165.1 (C═O), 154.8 (C═N), 152.6 (C═N), 134.7 (Ar—C), 132.6 (Ar—C), 124.2 (ipso-Ar—C), 120.1 (ipso-Ar—C); m/z (ES) 259 ([⁸¹Br]M), 257 ([⁷⁹Br]M); HRMS (ES⁻) Found M⁻, 256.90248 (C₇H₂⁷⁹BrN₂O₂S requires 256.90258).

### Standard Procedure for the Formation of Carboxamides, Carboxylates and Carbothioates

Diazole carboxylic acid (0.23 mmol) and a large excess of thionyl chloride were mixed and heated to 55° C. for 1 h. After cooling the solution was mixed with toluene and evaporated to dryness. The residue was then suspended in chloroform (4 ml) and treated with a large excess of amine, alcohol or thiol. The solution was stirred for 1 h and then evaporated to dryness. The residue was then subjected to flash chromatography (n-hexane/EtOAc 4:1, 7:3, 1:1) to afford the desired carboxamides.

### (2E,2′E)-Dibutyl 3,3′-(benzo[c][1,2,5]thiadiazole-4,7-diyl)diprop-2-enoate (JDS022-1) (Compound xxiv)

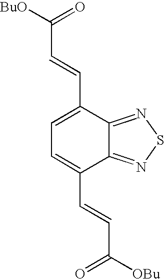

4,7-Dibromobenzo[c][1,2,5]thiadiazole (0.25 g, 0.85 mmol) and Pd(OAc)₂ (0.004 g, 0.017 mmol, 2 mol %) were dissolved in toluene (10 ml) and treated successively with butyl acrylate (0.12 ml, 0.85 mmol), DIPEA (0.33 ml, 1.87 mmol) and PPh₃ (0.004 g, 0.017 mmol, 2 mol %). The resulting solution was then refluxed for 24 h, cooled and poured in to H₂O. The aqueous layer was extracted with Et₂O (3×15 ml). The organic layers were dried over MgSO₄, filtered, concentrated and dried in vacuo. Flash chromatography (n-hexane/DCM [1:1], [3:7], [1:9], DCM) afforded the title compound as a dark green solid (0.05 g, 15%); R_{f} 0.2 (n-hexane/DCM 3:7); m.p. 90-94° C.; vmax(thin film) 2959, 2932, 2872, 1706 (C═O), 1631 (C═C), 1564, 1537, 1392, 1308, 1227, 1162, 983, 841 cm⁻¹; δH (200 MHz, CDCl₃) 8.02 (2H, d, J 16, CH═CH), 7.73 (2H, s, Ar—H), 7.52 (2H, d, J 16, CH═CH), 4.27 (2H, d, OCH₂CH₂CH₂CH₃), 1.80-1.73 (2H, m, OCH₂CH₂CH₂CH₃), 1.54-1.40 (2H, m, OCH₂CH₂CH₂CH₃), 0.98 (3H, m, OCH₂CH₂CH₂CH₃); δC (175 MHz, CDCl₃) 167.1 (C═O), 153.6 (q-C), 139.3 (C═C), 131.1 (Ar—C), 129.1 (q-C), 124.6 (C═C), 64.7 (OCH₂CH₂CH₂CH₃), 30.8 (OCH₂CH₂CH₂CH₃), 19.2 (OCH₂CH₂CH₂CH₃), 13.7 (OCH₂CH₂CH₂CH₃); m/z (EI) 332 (M⁺-Bu, 25%), 259 (M⁺-Bu, -BuO, 100%), 249 (80%).

### 4-Methylbenzo[c][1,2,5]selenadiazole (JDS062) (Compound xxv)

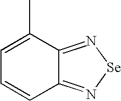

3-Methyl-1,2-phenylenediamine (2.0 g, 16.4 mmol) was dissolved in toluene (40 ml), treated with selenium oxychloride (1.1 ml, 16.4 mmol) dropwise and refluxed for 12 h. Once cooled to room temperature the solvent was removed and the residue subjected to flash chromatography (n-hexane, n-hexane/EtOAc 9:1, 4:1, 7:3) to afford the title compound as a light brown solid (1.42 g, 53%); R_{f} 0.7 (n-hexane/EtOAc 7:3); m.p. 90-94° C.; vmax(thin film) 1533, 1378, 1071, 1012, 859, 839, 794, 741, 705 cm⁻¹; δH (700 MHz, CDCl₃) 7.65 (1H, d, J 9, Ar—H), 7.35 (1H, dd, J 9, 6, Ar—H), 7.18 (2H, d, J 6, Ar—H), 2.68 (3H, s, Ar—CH₃); δC (175 MHz, CDCl₃) 161.4 (C═N), 161.0 (C═N), 133.3 (ipso-Ar—C), 130.1 (Ar—C), 128.0 (Ar—C), 121.4 (Ar—C), 18.4 (Ar—CH₃); m/z (EI) 198 ([⁷⁹Se]M⁺, 90%), 196 ([⁷⁷Se]M⁺, 50%), 170 (30%), 117 (M-⁷⁹Se, 45%), 91 (60%), 80 (50%), 64 (55%), 39 (100%); HRMS (EI) Found M⁺, 197.9691 (C₇H₆N₂⁷⁹Se requires 197.9691)

### 4-Bromo-7-methylbenzo[c][1,2,5]selenadiazole (JDS074) (Compound xxvi)

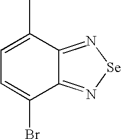

### Stage 1

4-Bromo-7-methylbenzo[c][1,2,5]thiadiazole (0.5 g, 2.2 mmol) was suspended in methanol (7 ml) and warmed to 45° C. (internal temperature). The suspension was then treated with magnesium turnings (0.42 g, 17.6 mmol) and stirred for 30 mins. The reaction was then cooled and the methanol removed in vacuo. The residue was then mixed with aq. NH₄Cl and extracted with EtOAc (3×30 ml). The organic layers were dried over MgSO₄, filtered, concentrated and dried in vacuo to afford 3-bromo-6-methylbenzene-1,2-diamine (0.39 g, 94%); δH (400 MHz, CDCl₃) 6.88 (1H, d, J 8, Ar—H), 6.47 (1H, d, J 8, Ar—H), 3.60 (4H, bs, Ar—NH₂), 2.16 (3H, s, ArCH₃); m/z (EI) 202 ([⁸¹Br]M⁺, 95%), 200 ([⁷⁹Br]M⁺, 100%), 120 (M⁺−Br, 75%); the resultant solid was used directly in the next stage.⁴⁶

### Stage 2

3-Bromo-6-methylbenzene-1,2-diamine (0.39 g, 2.1 mmol) was dissolved in toluene (10 ml) and treated with selenium oxychloride (0.14 ml, 2.1 mmol). The resultant suspension was refluxed overnight, cooled and the solvent removed in vacuo. The residue was then subjected to flash chromatography (CHCl₃) to afford the title compound as a yellow solid (0.27 g, 50%); R_{f} 0.4 (CHCl₃); m.p. 223-227° C.; vmax(thin film) 3025, 2911, 1595, 1476, 1372, 1071, 913, 831, 755, 710, 578 cm⁻¹; δH (700 MHz, CDCl₃) 7.65 (1H, d, J7, Ar—H), 7.12 (1H, dq, J7, 1, Ar—H), 2.65 (3H, d, J 1, Ar—CH₃); δC (176 MHz, CDCl₃) 160.4 (C═N), 158.1 (C═N), 132.9 (ipso-Ar—C), 132.2 (Ar—C), 128.2 (Ar—C), 114 (ispo-Ar—C), 18.1 (Ar—CH₃); m/z (EI) 278 ([⁸¹Br⁷⁹Se]M⁺, 30%), 276 ([⁷⁹Br⁷⁷Se, ⁷⁹Br⁷⁹Se]M⁺, 40%), 274 ([⁷⁹Br⁷⁷Se]M⁺, 20%), 197 (40%), 170 (40%), 117 (70%), 90 (100%); HRMS (EI) Found M⁺, 275.8796 (C₇H₅N₂⁸¹Br⁷⁷Se requires 275.8796)

### Benzo[c][1,2,5]selenadiazole-4,7-dicarbonitrile (JDS033) (Compound xxvii)

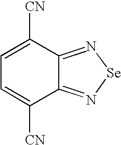

4,7-Dibromobenzo[c][1,2,5]selenadiazole (0.25 g, 0.74 mmol) and CuCN (0.07 g, 0.74 mmol) were dissolved in DMF (4 ml) and stirred at reflux for 2 h. The solution was then cooled and poured into NH₄OH and extracted with toluene (3×10 ml). The organic layers were dried over MgSO₄, filtered, concentrated and dried in vacuo to afforded the title compound as a beige solid (0.04 g, 25%); R_{f} 0.3 (DCM/n-hexane 6:4); vmax(thin film) 2235 (C═N), 877, 765, 634 cm⁻¹; δH (400 MHz, CDCl₃) 8.04 (2H, s, Ar—H); δC (126 MHz, CDCl₃) 156.6 (C═N—Se), 134.5 (Ar—C), 114.2 (CN), 112.4 (ipso-Ar—C); m/z (EI) 234 ([⁷⁹Se]M⁺, 100%), 232 (40%), 230 ([⁷⁶Se]M⁺, 25%); HRMS (EI) Found [⁷⁶Se]M⁺, 229.9463 (C₈H₂N₄⁷⁶Se requires 229.9466).

### 4,7-Dibromobenzo[c][1,2,5]oxadiazole (JDS0127)⁵² (Compound xxviii)

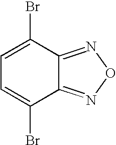

Benzo[c][1,2,5]oxadiazole (2.5 g, 21 mmol) was heated to 90° C. with iron powder (0.23 g, 4.2 mmol). The molten liquid was then treated with bromine (3.2 ml, 62 mmol) and refluxed for 2 h. The resultant solution was poured onto ice and the precipitate filtered. The precipitate was then mixed with aq. NaHCO₃, stirred for 15 min and filtered. Flash chromatography (n-hexane/EtOAc 9:1) afforded the title compound as an orange solid (1.5 g, 25%); m.p. 88-92° C.; vmax(thin film) 1514, 1344, 1201, 1026, 954, 871, 841 cm⁻¹; δH (500 MHz, CDCl₃) 7.51 (2H, s, Ar—H); 6C (126 MHz, CDCl₃) 149.6 (C═N), 134.4 (Ar—C), 108.9 (ipso-Ar—C); m/z (EI) 280 ([^{81,81}Br]M⁺, 6%), 278 ([^{81,79}Br]M⁺, 8%), 276 ([^{79,79}Br]M⁺, 6%), 250 (5%), 248 (10%), 246 (7%), 197 (5%); all data agree with those reported in the literature.

### 2-(7-Nitrobenzo[c][1,2,5]oxadiazol-4-yloxy)ethanol (JDS086-2)⁴⁸ (Compound xxix)

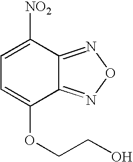

NBD-Cl (1.0 g, 5.0 mmol) was suspended in ethylene glycol (10 ml) and treated with a solution of NaOH (0.4 g, 10 mmol) in ethylene glycol (20 ml) at room temperature. The reaction mixture was stirred for 1 h and then acidified with 5 M HCl (20 ml). The resultant aqueous layer was extracted with EtOAc (3×20 ml). The organic layers were dried over MgSO₄, filtered, concentrated and dried in vacuo. Flash chromatography (CHCl₃/Acetone [9:1]) afforded the title compound as an orange oil (0.9 g, 80%); δH (400 MHz, CD₃OD) 8.64 (1H, d, J 8, Ar—H), 6.97 (1H, d, J 8, Ar—H), 4.50 (2H, t, J 4, OCH₂CH₂OH), 4.04 (2H, t, J 4, OCH₂CH₂OH); δC (126 MHz, CD₃OD) 153.9 (ipso-Ar—C), 144.6 (C═N), 143.3 (C═N), 133.9 (Ar—C), 128.6 (ipso-Ar—C), 104.3 (Ar—C), 71.6 (OCH₂CH₂OH), 58.7 (OCH₂CH₂OH); m/z (ES⁺) 248 (MNa⁺).

### Methyl 4-(7-bromobenzo[c][1,2,5]selenadiazol-4-yl) benzoate (Compound xxxi)

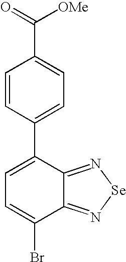

4,7-Dibromobenzo[c][1,2,5]selenadiazole (0.25 g, 0.74 mmol), 4-(methoxycarbonyl)phenylboronic acid (0.15 g, 0.74 mmol), Pd(PPh₃)₄ (0.014 g, 0.01 mmol) and Na₂CO₃ (0.09 g, 0.74 mmol) were weighed into a round bottom flask and dissolved with toluene (1 ml), THF (1 ml) and H₂O (0.2 ml). The solution was then refluxed for 24 h, cooled and poured in to H₂O. The aqueous layer was extracted with Et₂O (3×15 ml). The organic layers were dried over MgSO₄, filtered, concentrated and dried in vacuo. Flash chromatography (DCM/EtOAc [99:1], [98:2], [95:5]) afforded the title compound as a green solid (0.02 g, 7%); R_{f} 0.1 (n-hexane/DCM 1:1); δ_{H} (400 MHz, CDCl₃) 8.17 (1H, d, J 8, Ar—H), 7.90 (1H, d, J 8, Ar—H), 7.89 (1H, d, J 7, Ar—H), 7.47 (1H, d, J 7, Ar—H), 3.95 (3H, s, OCH₃).

### 7-Chloro-N,N-dimethylbenzo[c][1,2,5]oxadiazole-4-sulfonamide (Compound xxxiii)

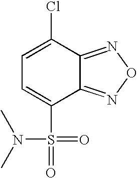

To a solution of 4-chloro-7-chlorosulfonyl-2,1,3-benzoxadiazole (0.127 g, 0.50 mmol) in anhydrous acetonitrile (6 mL) was added a solution of dimethylamine in THF (2.0 M, 0.30 mL, 0.60 mmol) followed immediately by triethylamine (0.15 mL, 1.10 mmol) under argon, with stirring. The reaction was stirred at room temperature for 10 min. before removing the solvent in vacuo. Purification by flash column chromatography on silica gel (PE 40-60:EtOAc, 8:2) gave the title product as a white solid (0.098 g, 0.38 mmol, 75%). ¹H NMR (500 MHz, CDCl₃): δ 7.97 (1H, d, J=7.3, H-5), 7.56 (1H, d, J=7.3, H-6), 2.96 (6H, s, 2×CH₃); ¹³C NMR (125 MHz, CDCl₃): δ 148.9 (C7a), 145.7 (C3a), 134.4 (C5), 129.1 (C6), 127.7 (C7), 126.1 (C4); MS-ES+ (m/z) 262 ([M+H]⁺, ³⁵C1, 100%); HRMS-ES+ (m/z) Calcd for C₈H₉N₃O₃³⁵Cl³²S [M+H]⁺: 262.0048, found 262.0048; Anal. Calcd for C₈H₈ClN₃O₃S: C, 36.72; H, 3.08; N, 16.06. Found: C, 37.11; H, 3.16; N, 15.85.

Compound xlvii) may be prepared by an analogous route to that used for compound x) replacing 4-(trifluoromethyl)thiophenol with 2-benzothiazolethiol (available from SinoChemexper Product List)

Compounds lvi) and lvii), may be prepared by reacting 2,1,3 benzothiadiazole-4-sulfonylchloride (commercially available from Apollo) with Hydrazinecarboxylic acid, 2-(aminoacetyl)-, 9H-fluoren-9-ylmethyl ester (Maybridge) deprotection of the Fmoc group and subsequent imine formation with an aldehyde (in this case 2-chlorobenzaldehyde)

Compound lvii), may be prepared by reacting 2,1,3 benzothiadiazole-4-sulfonylchloride (commercially available from Apollo) with Hydrazinecarboxylic acid, 2-(aminoacetyl)-, 9H-fluoren-9-ylmethyl ester (Maybridge) deprotection of the Fmoc group and subsequent acylation with an acid chloride (in this case with 2-chlorobenzoyl chloride)

Compound lx) may be prepared by combining the required piperazine (in this specific case 1-[2-(4-chlorophenyl)ethyl]-piperazine which is available from Aurora Screening Library) with 2,1,3 benzothiadiazole-4-sulfonylchloride (commercially available from Apollo)

Compound lxvi) may be prepared following analogous procedures as used to prepare compound xxxii) as found in Prados et al Analytica Chimica Acta 344 (1997) 227-232 in this case using as the phenolic component 2-chloro-4-trifluoromethylphenol.

An alternative procedure is found in the Central European Journal of Chemistry (2003), 1(3), 260-276.

Compound liii) may be prepared by combining excess amine (in this case morpholine) with 4-chloro-7-chlorosulfonyl-2,1,3-benzothiadiazole, which is described in J. Phys. Chem. B 2008, 112, 2829, in a similar fashion to that described for the preparation of compound xi).

### REFERENCES

1. I. Cummins, D. J. Cole, R. Edwards, Plant J. 18, 285 (1999).
2. D. P. Dixon, A. G. McEwen, A. J. Lapthorn, R. Edwards, J. Biol. Chem. 278, 23930 (2003).
3. S. J. Clough, A. F. Bent, Plant J. 16, 735 (1998)
4. I. Cummins, D. J. Cole, R. Edwards, Pestic. Biochem. Physiol. 59, 35 (1997).
5. I. Cummins, M. Brazier-Hicks, M. Stobiecki, R. Franski, R. Edwards, Phytochem. 67, 1722 (2006).
6. I. Cummins, R. Edwards Plant J. 39, 894 (2004).
7. D. P. Dixon, M. Skipsey, N. M. Grundy, R. Edwards, Plant Phys. 138, 2233 (2005).
8. F. L. Theodoulou, I. M. Clark, K. E. Pallett, D. L. Hallahan, Plant Physiol. 119, 1567 (1999).
9. D. P. Dixon, B. G. Davis, R. Edwards, J. Biol. Chem. 277, 30859 (2002).
10. H. Aebi, Methods Enz. 105, 121 (1984).
11. Y. Nakano, K. Asada, Plant Cell Physiol. 22, 867 (1981).
12. A. N. P. Hiner, J. N. Rodriguez-Lopez, M. B. Arnao, E. L. Raven, F. Garcia-Canovas, M. Acosta, Biochem. J. 348, 321 (2000).
13. H. Ukeda, S Maeda, T Ishii, M. Sawamura, Anal. Biochem. 251, 206 (1997).
14. I. K. Smith, T. L. Vierheller, C. A. Thorne, Anal. Biochem. 175, 408 (1988).
15. R. Edwards, J. W. Blount, R. A. Dixon, Planta 184, 403 (1991).
16. V. P. Roxas, S. A. Lodhi, D. K. Garrett, J. R. Mahan, R. D. Allen, Plant Cell Physiol. 41, 1229 (2000).
17. S. J. Bloor, S. Abrahams, Phytochem. 59, 343.
18. S. B. Powles, D. L. Shaner, ‘Herbicide Resistance and World Grains’ CRC Press (2001).
19. J. Gressel, ‘Molecular Biology of Weed Control’ Taylor and Francis (2002).
20. J. S. Yuan, P. J. Tranel, C. N. Stewart Jr., Trends Plant Sci., 12, 1360 (2006).
21. X-Q. Xhang, S. B. Powles, Planta 223, 550 (2006).
22. M. Sibony, B. Rubin, Planta 216, 1022 (2003).
23. P. J. Tranel, T. R. Wright, Weed Sci. 50, 700 (2002).
24. J. Menendez, R. DePrado, Pestic. Biochem. Physiol. 56, 123 (1996).
25. L. M. Hall, S. R. Moss, S. B. Powles, Pestic. Biochem. Physiol. 57, 87 (1997).
26. W. J. Owen, Herbicide metabolism as a basis for selectivity, in: T. Roberts (Ed.) Metabolism of Agrochemicals in Plants, Wiley, Chichester, UK. pp 211-258 (2000).
27. S. R. Moss, K. M. Cocker, A. C. Brown, L. Hall, L. M. Field, Pestic. Manag. Sci. 59, 190 (2003).
28. R. J Hyde, D. L. Hallahan, J. R. Bowyer Pestic. Sci. 47, 185 (1996).
29. I. Cummins, S. Moss, D. J. Cole, R. Edwards, Pestic. Sci 51, 244 (1997).
30. J. P. H. Reade, A. H. Cobb, Pestic. Sci. 55, 993 (1999).
31. M. Brazier, D. J. Cole, R. Edwards, Phytochem. 59, 149 (2002).
32. I. Cummins, D. J. Cole, R. Edwards, Plant J., 18, 285 (1999).
33. D. P. Dixon, B. G. Davis, R. Edwards, J. Biol. Chem. 277, 30859 (2002).
34. Materials and Methods are available as supporting material
35. F. L. Theodoulou, I. M. Clark, K. E. Pallett, D. L. Hallahan, Plant Physiol. 119, 1567 (1999).
36. Cummins, M. Brazier-Hicks, M. Stobiecki, R. Franski, R. Edwards, Phytochem. 67, 1722 (2006).
37. S. Mahajan, W. M. Atkins, Cell. Mol. Life. Sci. 62, 1221 (2005).
38. L. I. McLellan, R. Wolf, Drug Resist. Update 2, 153 (1999).
39. C. H. Foyer, G. Noctor, Plant Cell Env. 28, 1056 (2005).
40. N. H. P. Cnubben, I. M. C. M Rietjens, H. Wurtelboer, J. van Zanden, P. J. van Bladeren, Env. Toxicol Pharmacol. 10, 141 (2001).
41. L. Romero, K. Andrews, L. Ng, K O'Rourke, A. Maslen, G. Kirby, Biochem J. 400, 135 (2006).
42. P. Turella, C. Cerella, G. Fiomeni, A. Bullo, F. DeMaria, L. Ghibelli, M. R. Chiriolo, M. Cianfriglia, M. Mattei, G. Federici, G. Ricci, A. M. Caccuri, Cancer Res., 65, 3751 (2005).
43. Y. C. Aswasthi, Y. Yang, N. K. Tiwari, B. Patrick, A. Sharma, J. Li, S. Awasthi, Free Radical Biol. Med., 37, 607 (2004).
[44] Dalmonte, D.; Mazzaracchio, P.; Sandri, E., Boll. Sci. Fac. Chim. Ind. Bologna, 1968, 26, 165.
[45] Ahnoff, M.; Grundevik, I.; Arfwidsson, A.; Fonselius, J.; Persson, B.-A., Anal. Chem., 1981, 53, 485.
[46] Prashad, M.; Liu, Y.; Repic, O., Tetrahedron Lett., 2001, 42, 2277.
[47] Ricci, G.; De Maria, F.; Antonini, G.; Turella, P.; Bullo, A.; Stella, L.; Filomeni, G.; Federici, G.; Caccuri, A. M., J. Biol. Chem., 2005, 280, 26397.
[48] Johnson, L.; Lagerkvist, S.; Lindroth, P.; Ahnoff, M.; Martinsson, K., Anal. Chem., 1982, 54, 939.
[49] Pilgram, K.; Zupan, M., J. Org. Chem., 1971, 36, 207.
[50] Uchiyama, S.; Santa, T.; Fukushima, T.; Homma, H.; Imai, K., J. Chem. Soc., Perkin Trans. 2, 1998, 2165.
[51] PCT/US2002/040598
[52] Blouin, N.; Michaud, A.; Gendron, D.; Wakim, S.; Blair, E.; Neagu-Plesu, R.; Belletete, M.; Durocher, G.; Tao, Y.; Leclerc, M., J. Am. Chem. Soc., 2008, 130, 732.

What is claimed:

1.-37. (canceled)

38. A method for selectively controlling multiple herbicide resistance (MHR) in weed plants in the field, the method comprising i) applying to plants in the field at least one chemical inhibitor that is effective in regulating the enzymic activity of at least one glutathione transferase (GST) that is capable of conferring MHR to a plant or of at least one active subunit thereof, and ii) applying a herbicide.

39. A method according to claim 38 wherein the weed plants are of the Gramineae and/or of the Poaceae.

40. A method according to claim 39, wherein the GST is selected from the phi class of plant GST enzymes and active subunits thereof.

41. A method according to claim 40 wherein the GST is an AmGSTF1-1 or a functional homologue thereof.

42. A method for selectively controlling multiple herbicide resistance (MHR) in weed plants in the field according to claim 38, wherein the at least one chemical inhibitor is a compound of Formula (I) 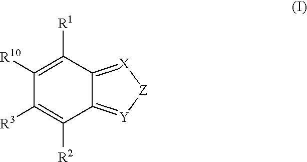
wherein
R¹ is selected from H, (C₁-C₁₅) alkyl, (C₁-C₁₋₅)haloalkyl, NO₂, SO₂NR⁴R⁵, SO₂R⁶, SO₂V, SO₂NH(CH₂)₁₋₆CONH—NHCOV, SO₂NH(CH₂)₁₋₆CONH—N═CHV, CHO, COOR⁷, CONR⁴R⁵, Br, Cl, F, CH═CHCOO(CH₂)ₙCH₃, CN,
SO₂(—NTN—)(CH₂)nV, SO₂N═SR⁸R⁹V, SO₂OV, COV, and (C₃-C₉) heteroaryl ring containing at least one of O, N, and S, C₆ aryl ring, C₁₀ aryl ring, wherein the said heteroaryl ring, said C₆ aryl ring and said C₁₀ aryl ring are optionally substituted with COOR⁷;
R² is selected from H, F, Cl, Br, (C₁-C₆)alkyl, (C₁-C₆)haloalkyl, NR⁴R⁵, OR⁴, SR⁴, S(CH₂)ₙOH, S(CH₂)ₙCOOR⁷, CH═CHCOOR⁷, CN, O(CH₂)ₙOR⁷, O(C₁-C₆)alkylCOOH, NHCO(C₁-C₆)alkyl, NHCO(C₆aryl), NHCO(C₁₀aryl), NHCO(heteroaryl ring), and a S(C₃-C₉) heteroaryl ring containing at least one of N, O, and S, optionally substituted with H, (C₁-C₆)alkyl, F, Br, C₁, NO₂, NHR⁴ or NR⁴R⁵;
R³ is selected from CF₃, NO₂ and H;
R⁴ and R⁵ are independently selected from H, (C₁-C₁₅) alkyl, (C₁-C₁₅) haloalkyl, (CH₂)ₙN₃, a C₆-aryl ring, a C₁₀aryl ring, a (C₃-C₉) heteroaryl ring containing at least one of O, S and N wherein the said C₆aryl ring, said C₁₀aryl ring, and said (C₃-C₉) heteroaryl ring are optionally substituted with at least one of OH, Cl, Br, F, CF₃, COO(C₁-C₆)alkyl, (C₁-C₆) alkyl; or R⁴ and R⁵ together form a 4-8 membered heterocyclic ring structure containing carbon atoms and optionally at least one ring member selected from O, S and N;
R⁶ is selected from H, (C₁-C₁₅) alkyl, OH, Cl, Br, and F;
R⁷ is selected from H, (C₁-C₆) alkyl, C₆-aryl ring, a C₁₀aryl ring, a (C₃-C₉) heteroaryl ring containing at least one of O, S and N wherein the said C₆aryl ring, said C₁₀aryl ring, and said (C₃-C₉) heteroaryl ring are optionally substituted with at least one of OH, Cl, Br, F, CF₃, COO(C₁-C₆)alkyl, and (C₁-C₆) alkyl;
R⁸ is ═O;
R⁹ is (C₁-C₆) alkyl;
R¹⁶ is selected from H, Cl, Br, F, (C₁-C₁₅) alkyl, (C₁-C₁₅) haloalkyl, SR⁴, and NR⁴R⁵;
(—NTN—) is a piperazine ring structure;
V is selected from a (C₃-C₉) heteroaryl ring containing at least one of O, S and N, a C₆aryl ring, a C₁₀aryl ring wherein the said (C₃-C₉) heteroaryl ring, said C₆ aryl ring and said C₁₀aryl ring are optionally substituted with at least one of (C₁-C₆) alkyl, CF₃, O, Br, Cl, and F;
X is selected from N and N⁺—O⁻;
Y is selected from N and N⁺—O⁻;
Z is selected from O, Se and S; and
n is a whole integer selected from 1 to 8.

43. A method according to claim 38 wherein the at least one chemical inhibitor is a compound of Formula (I) wherein:
R¹ is selected from H, (C₁-C₁₀) alkyl, (C₁-C₁₀) haloalkyl, NO₂, SO₂NR⁴R⁵, SO₂R⁶, SO₂V, SO₂NH(CH₂)₁₋₆CONH—NHCOV, SO₂NH(CH₂)₁₋₆CONH—N═CHV, CHO, COOR⁷, CONR⁴R⁵, Br, Cl, F, CH═CHCOO(CH₂)ₙCH₃, CN,
SO₂(—NTN—)(CH₂)nV, SO₂N═SR⁸R⁹V, SO₂OV, COV, and (C₄-C₇) heteroaryl ring containing at least one of O, N, and S, C₆ aryl ring, wherein the said heteroaryl ring, and said C₆ aryl ring are optionally substituted with COOR⁷;
R² is selected from H, F, Cl, Br, (C₁-C₆)alkyl (C₁-C₆)haloalkyl, NR⁴R⁵, OR⁴, S(CH₂)ₙOH, S(CH₂)ₙCOOR⁷, CH═CHCOOR⁷, CN, O(CH₂)ₙOR⁷, O(C₁-C₆)alkylCOOH, NHCO(C₁-C₆)alkyl, NHCO(C₆aryl), NHCO(C₁₀aryl), NHCO(heteroaryl ring), and a S(C₃-C₉) heteroaryl ring containing at least one of N, O, and S, optionally substituted with H, (C₁-C₆)alkyl, F, Br, C₁, NO₂, NHR⁴ or NR⁴R⁵;
R³ is selected from CF₃, NO₂ and H;
R⁴ and R⁵ are independently selected from H, (C₁-C₁₀) alkyl, (C₁-C₁₀) haloalkyl, (CH₂)ₙN₃, a C₆-aryl ring, a (C₄-C₈) heteroaryl ring containing at least one of O, S and N wherein the said C₆aryl ring, and said (C₄-C₈) heteroaryl ring are optionally substituted with at least one of OH, Cl, Br, F, CF₃, COO(C₁-C₆)alkyl, (C₁-C₆) alkyl; or R⁴ and R⁵ together form a 4 or 5 membered heterocyclic ring structure containing carbon atoms and optionally at least one ring member selected from O, S and N;
R⁶ is selected from H, (C₁-C₆) alkyl, OH, Cl, Br, and F;
R⁷ is selected from H, (C₁-C₆) alkyl, C₆-aryl ring, a (C₄-C₈) heteroaryl ring containing at least one of O, S and N wherein the said C₆aryl ring, and said (C₄-C₈) heteroaryl ring are optionally substituted with at least one of OH, Cl, Br, F, CF₃, COO(C₁-C₆)alkyl, and (C₁-C₆) alkyl;
R⁸ is ═O;
R⁹ is (C₁-C₆) alkyl;
R¹⁰ is selected from H, Cl, Br, F, (C₁-C₁₀) alkyl, (C₁-C₁₀) haloalkyl, SR⁴, and NH₂;
(—NTN—) is a piperazine ring structure;
V is selected from a (C₃-C₉) heteroaryl ring containing at least one of O, S and N, a C₆aryl ring, wherein the said (C₃-C₉) heteroaryl ring and said C₆ aryl ring are optionally substituted with at least one of (C₁-C₄) alkyl, CF₃, O, Br, Cl, and F;
X is selected from N and N⁺—O⁻;
Y is selected from N and N⁺—O⁻;
Z is selected from O, Se and S; and
n is a whole integer selected from 1 to 8.

44. A method for selectively controlling multiple herbicide resistance (MHR) in weed plants in a field according to claim 38, wherein the at least one chemical inhibitor is a compound of Formula (I) 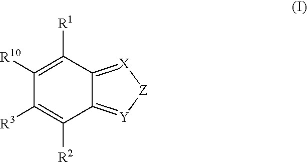
wherein
R¹ is selected from H, (C₁-C₆) alkyl, (C₁-C₆) haloalkyl, NO₂, SO₂NR⁴R⁵, SO₂R⁶, SO₂V,
SO₂NH(CH₂)₁₋₄CONH—NHCOV, SO₂NH(CH₂)₁₋₄CONH—N═CHV, CHO, COOR⁷, CONR⁴R⁵, Br, Cl, F, CH═CHCOO(CH₂)CH₃, CN, SO₂(—NTN—)(CH₂)nV, SO₂N═SR⁸R⁹V, SO₂OV, COV, and (C₄-C₇) heteroaryl ring containing at least one of O, N, and S, C₆ aryl ring, wherein the said heteroaryl ring, and said C₆ aryl ring are optionally substituted with COOR⁷;
R² is selected from H, F, Cl, Br, (C₁-C₆)alkyl, (C₁-C₆)haloalkyl, NR⁴R⁵, OR⁴, S(CH₂)ₙOH, S(CH₂)ₙCOOR⁷, CH═CHCOOR⁷, CN, O(CH₂)ₙOR⁷, O(C₁-C₆)alkylCOOH,
NHCO(C₁-C₆)alkyl, NHCO(C₆aryl), NHCO(heteroaryl ring), and a S(C₃-C₉) heteroaryl ring containing at least one of N, O, and S, optionally substituted with H, (C₁-C₆)alkyl, F, Br, C₁, NO₂, NHR⁴ or NR⁴R⁵;
R³ is selected from CF₃, NO₂ and H;
R⁴ and R⁵ are independently selected from H, (C₁-C₆) alkyl, (C₁-C₆) haloalkyl, (CH₂)ₙN₃, a C₆-aryl ring, a (C₄-C₈) heteroaryl ring containing at least one of O, S and N wherein the said C₆aryl ring, and said (C₄-C₈) heteroaryl ring are optionally substituted with at least one of OH, Cl, Br, F, CF₃, COO(C₁-C₆)alkyl, (C₁-C₆) alkyl; or R⁴ and R⁵ together form a 4 or 5 membered heterocyclic ring structure containing carbon atoms and optionally at least one ring member selected from O, S and N;
R⁶ is selected from H, (C₁-C₆) alkyl, OH, Cl, Br, and F;
R⁷ is selected from H, (C₁-C₄) alkyl, C₆-aryl ring, a (C₄-C₈) heteroaryl ring containing at least one of O, S and N wherein the said C₆aryl ring, and said (C₄-C₈) heteroaryl ring are optionally substituted with at least one of OH, Cl, Br, F, CF₃, COO(C₁-C₄)alkyl, and (C₁-C₄) alkyl;
R⁸ is ═O;
R⁹ is (C₁-C₆) alkyl;
R¹⁰ is selected from H, Cl, Br, F, (C₁-C₁₀) alkyl, (C₁-C₁₀) haloalkyl, SR⁴, and NH₂;
(—NTN—) is a piperazine ring structure;
V is selected from a (C₃-C₇) heteroaryl ring containing at least one of O, S and N, a C₆aryl ring, wherein the said (C₃-C₉) heteroaryl ring and said C₆ aryl ring are optionally substituted with at least one of (C₁-C₄) alkyl, CF₃, O, Br, Cl, and F;
X is selected from N and N⁺—O⁻;
Y is selected from N and N⁺—O⁻;
Z is selected from O, Se and S; and
n is a whole integer selected from 1 to 6.

45. A method for selectively controlling multiple herbicide resistance (MHR) in weed plants in the field according to claim 38 that comprises applying to plants in the field at least one chemical inhibitor of Formula (I) 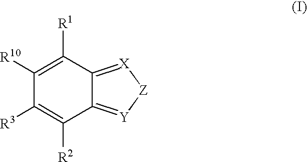
wherein:
R¹ is selected from H, (C₁-C₃) alkyl, (C₁-C₃) haloalkyl, NO₂, SO₂NR⁴R⁵, SO₂R⁶, SO₂V, SO₂NH(CH₂)₁₋₄CONH—NHCOV, SO₂NH(CH₂)₁₋₄CONH—N═CHV, CHO, COOR⁷, CONR⁴R⁵, Br, Cl, F, CH═CHCOO(CH₂)₄CH₃, CN,
SO₂(—NTN—)(CH₂)nV, SO₂N═SR⁸R⁹V, SO₂OV, COV, and (C₄-C₇) heteroaryl ring containing at least one of O, N, and S, C₆ aryl ring, wherein the said heteroaryl ring, and said C₆ aryl ring are optionally substituted with COOR⁷;
R² is selected from H, F, Cl, Br, (C₁-C₃)alkyl, (C₁-C₃)haloalkyl, NR⁴R⁵, OR⁴, SR⁴, S(CH₂)₆OH, S(CH₂)₂COOR⁷, CH═CHCOOR⁷, CN, O(CH₂)₂OR⁷, O(C₁-C₆)alkylCOOH, NHCO(C₁-C₆)alkyl, NHCO(C₆aryl), NHCO(heteroaryl ring), and a S(C₃-C₉) heteroaryl ring containing at least one of N, O, and S, optionally substituted with H, (C₁-C₆)alkyl, F, Br, C₁, NO₂, NHR⁴ or NR⁴R⁵;
R³ is selected from CF₃, NO₂ and H;
R⁴ and R⁵ are independently selected from H, (C₁-C₄) alkyl, (C₁-C₄) haloalkyl, (CH₂)₄N₃, a C₆-aryl ring, a (C₄-C₈) heteroaryl ring containing at least one of O, S and N wherein the said C₆aryl ring, and said (C₄-C₈) heteroaryl ring are optionally substituted with at least one of OH, Cl, Br, F, CF₃, COO(C₁-C₆)alkyl, (C₁-C₆) alkyl; or R⁴ and R⁵ together form a 4 or 5 membered heterocyclic ring structure containing carbon atoms and optionally at least one ring member selected from O, S and N;
R⁶ is selected from H, (C₁-C₆) alkyl, OH, Cl, Br, and F;
R⁷ is selected from H, (C₁-C₄) alkyl, C₆-aryl ring, a (C₄-C₈) heteroaryl ring containing at least one of O, S and N wherein the said C₆aryl ring, and said (C₄-C₈) heteroaryl ring are optionally substituted with at least one of OH, Cl, Br, F, CF₃, COO(C₁-C₃)alkyl, and (C₁-C₄) alkyl;
R⁸ is ═O;
R⁹ is (C₁-C₆) alkyl;
R¹⁰ is selected from H, Cl, Br, F, (C₁-C₆) alkyl, (C₁-C₆) haloalkyl, SR⁴, and NH₂;
(—NTN—) is a piperazine ring structure;
V is selected from a (C₃-C₇) heteroaryl ring containing at least one of O, S and N, a C₆aryl ring, wherein the said (C₃-C₉) heteroaryl ring and said C₆aryl ring are optionally substituted with at least one of CF₃, O, Br, Cl, and F;
X is selected from N and N⁺—O⁻;
Y is selected from N and N⁺—O⁻;
Z is selected from O, Se and S; and
n is a whole integer selected from 1 to 6.

46. A method according to claim 38 for selectively controlling multiple herbicide resistance (MHR) in weed plants in the field comprises applying to plants in the field at least one chemical inhibitor of Formula (I) 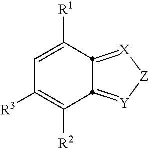
Wherein
R¹ is selected from H, (C₁-C₃) alkyl, (C₁-C₃) haloalkyl, NO₂, SO₂NR⁴R⁵, SO₂R⁶, SO₂V, SO₂NH(CH₂)₁₋₄CONH—NHCOV, SO₂NH(CH₂)₁₋₄CONH—N═CHV, CHO, COOR⁷, CONR⁴R⁵, Br, Cl, F, CH═CHCOO(CH₂)₄CH₃, CN,
SO₂(—NTN—)(CH₂)nV, SO₂N═SR⁸R⁹V, SO₂OV, COV, and (C₄-C₇) heteroaryl ring containing at least one of O, N, and S, C₆ aryl ring, wherein the said heteroaryl ring, and said C₆ aryl ring are optionally substituted with COOR⁷;
R² is selected from H, F, Cl, Br, (C₁-C₃)alkyl, (C₁-C₃)haloalkyl, NR⁴R⁵, OR⁴, SR⁴, S(CH₂)₆OH, S(CH₂)₂COOR⁷, CH═CHCOOR⁷, CN, O(CH₂)₂OR⁷, O(C₁-C₆)alkylCOOH, NHCO(C₁-C₆)alkyl, NHCO(C₆aryl), NHCO(heteroaryl ring), and a S(C₃-C₉) heteroaryl ring containing at least one of N, O, and S, optionally substituted with H, (C₁-C₆)alkyl, F, Br, C₁, NO₂, NHR⁴ or NR⁴R⁵;
R³ is selected from CF₃, NO₂ and H;
R⁴ and R⁵ are independently selected from H, (C₁-C₄) alkyl, (C₁-C₄) haloalkyl, (CH₂)₄N₃, a C₆-aryl ring, a (C₄-C₈) heteroaryl ring containing at least one of O, S and N wherein the said C₆aryl ring, and said (C₄-C₈) heteroaryl ring are optionally substituted with at least one of OH, Cl, Br, F, CF₃, COO(C₁-C₆)alkyl, (C₁-C₆) alkyl; or R⁴ and R⁵ together form a 4 or 5 membered heterocyclic ring structure containing carbon atoms and optionally at least one ring member selected from O, S and N;
R⁶ is selected from H, (C₁-C₆) alkyl, OH, Cl, Br, and F;
R⁷ is selected from H, (C₁-C₄) alkyl, C₆-aryl ring, a (C₄-C₈) heteroaryl ring containing at least one of O, S and N wherein the said C₆aryl ring, and said (C₄-C₈) heteroaryl ring are optionally substituted with at least one of OH, Cl, Br, F, CF₃, COO(C₁-C₃)alkyl, and (C₁-C₄) alkyl;
R⁸ is ═O;
R⁹ is (C₁-C₆) alkyl;
(—NTN—) is a piperazine ring structure;
V is selected from a (C₃-C₇) heteroaryl ring containing at least one of O, S and N, a C₆aryl ring, wherein the said (C₃-C₉) heteroaryl ring and said C₆aryl ring are optionally substituted with at least one of CF₃, O, Br, Cl, and F;
X is selected from N and N⁺—O⁻;
Y is selected from N and N⁺—O⁻;
Z is selected from O, Se and S; and
n is a whole integer selected from 1 to 6.

47. A method according to claim 46 wherein the at least one chemical compound is selected from the group:
Compound i) 4-Chloro-7-nitrobenzo[c][1,2,5]oxadiazole;
Compound ii) 4-Methoxy-7-nitrobenzo[c][1,2,5]oxadiazole;
Compound iii) 4-Nitro-7-(pyrrolidin-1-yl)benzo[c][1,2,5]oxadiazole;
Compound iv) 7-Chloro-N-methylbenzo[c][1,2,5]oxadiazole-4-sulfonamide;
Compound v) N-(4-azidobutyl)-7-chlorobenzo[c][1,2,5]oxadiazole-4-sulfonamide;
Compound vi) 4-Fluoro-7-nitrobenzo[c][1,2,5]oxadiazole;
Compound vii) 4,6-Dinitrobenzo[c][1,2,5]oxadiazole 1-oxide;
Compound viii) 4-bromo-7-nitrobenzo[c][1,2,5oxadiazole;
Compound ix) 4-(methylthio)-7-nitrobenzo[c][1,2,5oxadiazole;
Compound x) 4-Nitro-7-(4-trifluoromethyl)phenylthio)benzo[c][1,2,5oxadiazole;
Compound xi) 4-morpholino-7-nitrobenzo[c][1,2,5oxadiazole;
Compound xii) 4-ethoxy-7-nitrobenzo[c][1,2,5oxadiazole;
Compound xiii) 7-Nitrobenzo[c][1,2,5oxadiazole;
Compound xiv) 3-(7-Nitrobenzo[c][1,2,5oxadiazol-4-ylthio)propanoic acid;
Compound xv) 6-(7-Nitrobenzo[c][1,2,5oxadiazol-4-ylthio)hexan-1-ol;
Compound xvi) 7-bromo-N-propylbenzo[c][1,2,5thiadiazole-4-carboxamide;
Compound xvii) 7-bromo-N-methylbenzo[c][1,2,5thiadiazole-4-carboxamide;
Compound xviii) 7-bromo-N,N-dimethylbenzo[c][1,2,5 thiadiazole-4-carboxamide;
Compound xix) Methyl 7-bromobenzo[c][1,2,5thiadiazole-4-carboxylate;
Compound xx) Methyl 4-(7-bromobenzo[c][1,2,5thiadiazole-4-yl)benzoate;
Compound xxi) 4-Bromo-7-nitrobenzo[c][1,2,5thiadiazole;
Compound xxii) 4-Bromo-7-nitrobenzo[c][1,2,5selenadiazole;
Compound xxiii) 7-Bromobenzo[c][1,2,5thiadiazole-4-carboxylic acid;
Compound xxiv) (2E,'2E)-Dibutyl 3,3′-(benzo[c][1,2,5thiadiazole-4,7-diprop-2-enoate;
Compound xxv) 4-Methylbenzo[c][1,2,5selenadiazole;
Compound xxvi) 4-Bromo-7-methylbenzo[c][1,2,5selenadiazole;
Compound xxvii) Benzo[c][1,2,5selenadiazole-4,7-dicarbonitrile;
Compound xxviii) 4,7-Dibromobenzo[c][1,2,5oxadiazole;
Compound xxix) 2-(7-nitrobenzo[c][1,2,5oxadiazol-4-yloxy)ethanol;
Compound xxx) 4-Nitrobenzo[c][1,2,5oxadiazole;
Compound xxxi) Methyl 4-(7-bromobenzo[c][1,2,5selenadiazol-4-yl)benzoate;
Compound xxxii) 4-Nitro-7-phenoxybenzo[c][1,2,5oxadiazole;
Compound xxxiii) 7-Chloro-N,N-dimethylbenzo[c][1,2,5oxadiazole-4-sulfonamide;
Compound xxxiv) 4-Bromobenzo[c][1,2,5oxadiazole;
Compound xxxv) 4-Nitro-7-(piperidin-1-yl)benzo[c][1,2,5oxadiazole;
Compound xxxvi) 5-Chloro-4-nitrobenzo[c][1,2,5thiadiazole;
Compound xxxvii) 7-Chloro-4-nitrobenzo[c][1,2,5oxadiazole 1-oxide;
Compound xxxviii) 4-Nitro-7-phenoxybenzo[c][1,2,5oxadiazole;
Compound xxxix) N-Methyl-7-nitrobenzo[c][1,2,5oxadiazol-4-amine;
Compound xl) N-(7-Nitrobenzo[c][1,2,5oxadiazol-4-yl)ethanamide;
Compound xli) 4-(Methyl(7-nitrobenzo[c][1,2,5oxadiazol-4-yl)amino)phenyl;
Compound xlii) 7-Methyl-4-nitrobenzo[c][1,2,5oxadiazole 1-oxide;
Compound xliii) 5,7-Dinitrobenzo[c][1,2,5oxadiazole 1-oxide;
Compound xliv) 4-(5,7-Dinitrobenzo[c][1,2,5oxadiazol-4-ylamino)phenol;
Compound xlv) Methyl 4-(5,7-dinitrobenzo[c][1,2,5oxadiazol-4-ylamino)benzoate;
Compound xlvi) 7-Bromo-5-methyl-4-nitrobenzo[c][1,2,5oxadiazole;
Compound xlvii) N,N-dipropylbenzo[c][1,2,5thiadiazole-4-sulfonamide;
Compound xlviii) 4-(Benzo[d]thiazol-2-ylthio)-7-nitrobenzo[c][1,2,5thiadiazole;
Compound xlix) Bis(7-nitrobenzo[c][1,2,5thiadiazol-4-yl)sulfane;
Compound l) 5-(4-Chlorophenylthio)-4-nitrobenzo[c][1,2,5thiadiazole;
Compound li) 5,7-Dinitrobenzo[c][1,2,5thiadiazol-4-amine;
Compound lii) 5-Chloro-4-nitrobenzo[c][1,2,5selenadiazole;
Compound liii) 4-(7-Morpholinobenzo[c][1,2,5thiadiazol-4-ylsulfonyl)morpholine;
Compound liv) 4-Nitrobenzo[c][1,2,5thiadiazole;
Compound lv) (E)-N-(2-(2-(4-Chlorobenzylidene)hydrazinyl)-2-oxoethyl)benzo[c][1,2,5thiadiazole-4-sulfonamide;
Compound lvi) (E)-N-(2-(2-(2-Chlorobenzylidene)hydrazinyl)-2-oxoethyl)benzo[c][1,2,5thiadiazole-4-sulfonamide;
Compound lxvii) N-(2-(2-(2-Chlorophenylcarbonyl)hydrazinyl)-2-oxoethyl)benzo[c][1,2,5thiadiazole-4-sulfonamide;
Compound lviii) N-(2-(2-(4-Chlorophenylcarbonyl)hydrazinyl)-2-oxoethyl)benzo[c][1,2,5thiadiazole-4-sulfonamide;
Compound lix) N-(2-Oxo-2-(2-(3 trifluoromethyl)phenylcarbonyl)hydrazinyl)ethyl)benzo[c][1,2,5thiadiazole-4-sulfonamide;
Compound lx) 4-(4-(4-Chlorophenethyl)piperazin-1-ylsulfonyl)benzo[c][1,2,5thiadiazole-4-sulfonamide;
Compound lxi) S-Methyl-5-phenyl-N-(benzo[c][1,2,5oxadiazolyl-4-sulfonyl)sulfoximine;
Compound lxii) 3,5-Dichlorophenylbenzo[c][1,2,5thiadiazole-4-sulfonate;
Compound lxiii) 4-Chlorophenyl benzo[c][1,2,5thiadiazole-4-sulfonate;
Compound lxiv) 4-Nitrobenzo[c][1,2,5oxadiazol-5-amine;
Compound lxv) 4-(2-Chloro-4-(trifluoromethyl)phenoxy)-7-nitrobenzo[c]-[1,2,5oxadiazole;
Compound lxvi) 7-Nitro-N,N-dipropylbenzo[c][1,2,5oxadiazol-4-amine; and
Compound lxvii) Benzo[c][1,2,5oxadiazole.

48. A method according to claim 38 wherein the weed plant is a species of the Graminae or the Poaceae.

49. A method according to claim 38 wherein the weed plant is a plant from the Echinochloa, Setaria, Sorghum, Phalaris or Bromus families.

50. A method according to claim 38 wherein the weed plant is selected from black-grass (Alopecurus myosuroides), wild oat (Avera fatua), or annual rye-grass (Lolium rigidum)

51. A method according to claim 38 wherein the herbicide is selected from graminicides.

52. A method according to claim 51 wherein the graminicide is selected from the aryloxyphenoxypropionate class, phenyl urea class, triazine class, sulfonyl urea class, and cyclohexanedione class of graminicides.

53. A method according to claim 52 wherein the graminicide is selected from chlortoluron, fenoxapropethyl, pinoxaden, iodosulfuron methyl, atrazine, flufenacet, pendimethalin, prosulfocarb and triallate.

54. A method for selectively controlling the viability of plants displaying MHR in a field that comprises:
i) contacting the said plants with a chemical inhibitor of a GST that confers GST-mediated MHR to the plants; and
ii) contacting the said plants with at least one herbicide.

55. A method according to claim 54 that comprises:
i) contacting the said plants with at least one chemical inhibitor of a GST of Formula (I) according to claim 40 that confers GST-mediated MHR to the plants; and
ii) contacting the said plants with at least one herbicide.

56. A method according to claim 54 that comprises:
i) contacting the said plants with at least one chemical inhibitor of a GST of Formula (I) according to claim 40 selected from compounds i) to vii) that confers GST-mediated MHR to the plants; and
ii) contacting the said plants with at least one herbicide.

57. A method according to claim 54 wherein the said chemical inhibitor is applied before application of herbicide.

58. A method according to claim 55 wherein the said chemical inhibitor is applied before application of herbicide.

59. A method according to claim 56 wherein the said chemical inhibitor is applied before application of herbicide.

60. Use of a chemical inhibitor according to Formula (I) of claim 42 in a method for selectively controlling GST activity in MHR weed plants.

61. Use of a chemical inhibitor according to Formula (I) of claim 42 in a method for selectively controlling non-native GST activity in a transformed crop plant that comprises a non-native GST species that confers MHR thereto.

62. Use of a chemical inhibitor according to Formula (I) of claim 42 wherein the chemical inhibitor is a 2,1,3-benzoxadiazole.

63. Use of a chemical inhibitor according to Formula (I) of claim 47 wherein the chemical inhibitor is selected from compounds i) to lxviii).

64. A compound of Formula (Ia); 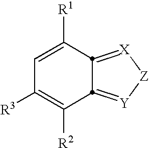
Wherein
R¹ selected from NO₂, CONR⁴R⁵, CN, SO₂NR⁴R⁵, COOR⁴, CONR⁴R⁵, and a C₆aryl ring optionally substituted with COOR⁴;
R² is selected from H, F, Cl, Br, CN, NHR⁴, NR⁴R⁵, OR⁴, and SR⁴;
R³ is selected from NO₂ and H;
R⁴ and R⁵ are independently selected from H, (C₁-C₄) alkyl, (CH₂)ₙN₃ or a C₆aryl ring optionally substituted with CF₃, or a C₆-aryl ring, a (C₄-C₇) heteroaryl ring containing at least one of O, S and N wherein the said C₆aryl ring, and said (C₄-C₇) heteroaryl ring are optionally substituted with at least one of OH, Cl, Br, F, CF₃, COO(C₁-C₆)alkyl, (C₁-C₆) alkyl; or R⁴ and R⁵ together faun a 5 membered heterocyclic ring structure containing carbon atoms and optionally at least one ring member selected from O, S and N;
X is selected from N and N⁺—O⁻;
Y is selected from N and N⁺—O⁻;
Z is selected from O, Se and S.

65. A compound according to claim 64 which is selected from the group of compounds iv), v), xvi)-xx), xxvii) and xxxi).

66. A method for identifying a GST inhibitor for use in the control of MHR weed plants in a field comprising i) isolating a plant cell from a plant that displays MHR ii) applying an organic chemical to the plant cell; iii) applying at least one class of herbicide to the said plant cell; and iv) analysing the said plant cell for viability.

67. A method according to claim 66 wherein the organic chemical of step ii) is selected from a compound of Formula (I) according to claim 42.

68. A method for identifying a GST inhibitor for use in the control of MHR weed plants in a field according to claim 66 that comprises applying at least two herbicides having different modes of action to the said plant cell; and iv) analysing the said plant cell for viability.

69. A method for identifying a GST inhibitor for use in the control of MHR weed plants in a field according to claim 67 that comprises applying at least two herbicides having different modes of action to the said plant cell; and iv) analysing the said plant cell for viability.

70. A method for identifying a GST inhibitor for use in the control of MHR weed plants in a field according to claim 66 that comprises i) isolating a plant cell from a plant that displays MHR; ii) applying an organic chemical to the plant cell; iii) applying a first herbicide having a first mode of action to the said plant cell; iv) analysing the said plant cell for viability; v) adding a second herbicide having a mode of action different to that of the first herbicide to a viable plant cell obtained from step iv); and vi) analyzing the plant cell for viability.

71. A method for identifying a GST inhibitor by screening an isolated GST derived from a plant that displays MHR that comprises i) measuring GST activity; ii) contacting an organic chemical compound with the isolated GST; iii) measuring GST activity after contact with the said chemical compound; and iv) comparing the GST activity measured under step i) with that of step iii).

72. A method for identifying a GST inhibitor by screening an isolated GST derived from a plant that displays MHR that comprises i) measuring GST activity; ii) contacting an organic chemical compound wherein the organic chemical is selected from a compound of Formula (I) according to claim 42 with the isolated GST; iii) measuring GST activity after contact with the said chemical compound; and iv) comparing the GST activity measured under step i) with that of step iii).

73. A method according to claim 66 wherein the at least one herbicide is selected from graminicidal herbicides selected from the aryloxyphenoxypropionate class, phenyl urea class, triazine class, sulfonyl urea class, and cyclohexanedione class of graminicides.

74. A method according to claim 67 wherein the at least one herbicide is selected from graminicidal herbicides selected from the aryloxyphenoxypropionate class, phenyl urea class, triazine class, sulfonyl urea class, and cyclohexanedione class of graminicides.

75. A method according to claim 68 wherein the at least one herbicide is selected from graminicidal herbicides selected from the aryloxyphenoxypropionate class, phenyl urea class, triazine class, sulfonyl urea class, and cyclohexanedione class of graminicides.

76. A method according to claim 69 wherein the at least one herbicide is selected from graminicidal herbicides selected from the aryloxyphenoxypropionate class, phenyl urea class, triazine class, sulfonyl urea class, and cyclohexanedione class of graminicides.

77. A method according to claim 70 wherein the at least one herbicide is selected from graminicidal herbicides selected from the aryloxyphenoxypropionate class, phenyl urea class, triazine class, sulfonyl urea class, and cyclohexanedione class of graminicides.

78. A method according to claim 71 wherein the at least one herbicide is selected from graminicidal herbicides selected from the aryloxyphenoxypropionate class, phenyl urea class, triazine class, sulfonyl urea class, and cyclohexanedione class of graminicides.

79. A method according to claim 72 wherein the at least one herbicide is selected from graminicidal herbicides selected from the aryloxyphenoxypropionate class, phenyl urea class, triazine class, sulfonyl urea class, and cyclohexanedione class of graminicides.

80. A method according to claim 73 wherein the at least one graminicidal herbicide is selected from chlortoluron, fenoxapropethyl, pinoxaden, iodosulfuron methyl, atrazine, flufenacet, pendimethalin, prosulfocarb and triallate.

81. A composition comprising at least one compound of Formula (I) of claim 42 together with excipients, diluents and/or additives for use in a method according to said claim 42.

82. A composition according to claim 81 wherein the at least one compound of Formula (I) is selected from compounds i) to lxviii).

83. A composition according to claim 81 that comprises at least two compounds of Formula (I).

84. A composition according to claim 83 that comprises at least two compounds that are selected from compounds i) to lxviii). of Formula (I).
